(19) United States
(12) Patent Application Publication
(10) Pub. No.: US 2022/0153791 A1
(43) Pub. Date: May 19, 2022

(54) EPHB4-EPHRIN B2 RECEPTOR LIGAND PAIR AS A NOVEL MARKER FOR THE TREATMENT OF PROSTATE CANCER

(71) Applicant: Vasgene Therapeutics Inc, Los Angeles, CA (US)

(72) Inventor: Valery Krasnoperov, Pasadena, CA (US)

(21) Appl. No.: 17/430,186

(22) PCT Filed: Feb. 13, 2020
(86) PCT No.: PCT/US20/18160

§ 371 Date: Aug. 11, 2021

Related U.S. Application Data
(60) Provisional application No. 62/805,291, filed on Feb. 13, 2019.

(51) Int. Cl. C07K 14/47; A61P 35/00

(52) U.S. Cl. CPC C07K 14/4703; A61K 38/00; A61P 35/00

(57) ABSTRACT

Compositions and methods are provided for treating prostate cancer (PC) in a subject comprising administering to the subject a therapeutically effective amount of a polypeptide agent that inhibits EphB4 or Ephrin B2 mediated functions. More specifically, methods are provided for use in treating PTEN deficient PC or PC that is refractory to treatment using androgen receptor (AR) targeted therapy. Importantly, a therapeutic agent, soluble EphB4, prevented tumor formation and induced tumor regression in established pre-castration and post-castration tumors. Surprisingly, androgen receptor (AR) levels also declined with therapy. PI3K isoform analysis showed downregulation of only PI3K p110 beta which directly regulates AR levels, such that AR decline was rescued with ectopic expression of PI3K beta. EphB4 is thus a novel target in prostate cancer.

### RELATED PATENT APPLICATIONS

This application claims benefit of U.S. Provisional Application No. 62/805,291, filed on Feb. 13, 2019, incorporated in its entirety by reference herein.

### SEQUENCE LISTING

The instant application contains a Sequence Listing in the form of a “paper copy” (PDF File) and a file containing the referenced sequences (SEQ ID NOS: 1-2) in computer readable form (ST25 format text file) which is submitted herein. The Sequence Listing is shown using standard three letter code for amino acids, as defined in 37 C.F.R. 1.822.

### TECHNICAL FIELD

Today, cancer remains a major cause of death worldwide despite the numerous advanced diagnostic and therapeutic methods that have been developed. Curative treatment protocols in clinical oncology remain reliant upon a combination of surgical resection, ionizing radiation, and cytotoxic chemotherapy. The major barrier to successful treatment and prevention of cancer lies in the fact that many cancers still fail to respond to the current chemotherapeutic and immunotherapy intervention, and many individuals suffer a recurrence or death, even after aggressive therapy. To address these shortcomings, there has been a trend in drug discovery to develop targeted therapies capable of modulating signaling axes dysregulated in cancers. There are now many FDA approved antibodies and small molecules that allow for therapeutic manipulation of a myriad of clinically relevant targets.

Prostate cancer (PC) is the most common non-skin cancer in men, and the second most common cause of death from cancer in men so that around 30,000 patients die each year in the US alone from metastatic disease. Androgen deprivation therapy (ADT), which has been a common strategy for treating advanced prostate cancer, induces significant regression of prostate tumors. However, while ADT initially achieves therapeutic response, it eventually fails in nearly all patients. Consequently, the patients develop castration resistant prostate cancer (CRPC) that is the invariable recurrence of aggressive, lethal prostate cancer in an androgen-depleted setting within two to three years after initiating therapy. Unfortunately, CRPC is incurable to date and almost every patient with metastatic CRPC eventually succumbs to the disease. Despite many different medications that have been developed and applied to patients since then, the fundamental premise behind androgen deprivation has remained almost unchanged. More than 250,000 men die from lethal prostate cancer worldwide each year. Therefore, therapeutic options for the patients are urgently needed. The disclosure is directed to this, as well as other, important ends.

Next generation sequencing has identified mutations in the phosphatidylinositol-4,5-bisphosphate 3-kinase (PI3K) pathway including mutations in PI3KCA and phosphatase and tensin homolog (PTEN) as early events in PC (Bezinelli et al., Bone Marrow Transplant, 52(10), 1384-1389, 2017). Although investigation of PI3K isoform specific inhibitors are currently in clinical trials, PI3K pathway inhibitors have had limited activity to this point. As such, identification of PC dominant drivers and development of novel therapies impacting these crucial targets are needed.

Eph (Erythropoietin Producing Hepatoma) receptor and ligand are part of the largest family of receptor tyrosine kinases (RTKs). The family is subdivided into class A and class B, based on sequence homology and binding affinity for two distinct types of membrane-anchored ephrin ligands. Each Eph receptor and ligand can bind to multiple ligands and receptors and certain receptors have been postulated as putative tumor suppressors and others as tumor promoters (Vaught et al, Breast Cancer Res, 10(6):217-224, 2008). EphB4 and ephrin B2 function as a tyrosine kinase receptor-ligand pair, which is found primarily on endothelial cells and are involved in vasculogenesis and angiogenesis. Inhibition of the Ephrin B2-EphB4 interaction has a direct inhibitory effect on tumor cell proliferation in vitro and ex-vivo. Polypeptide agents that inhibit EphB4 or Ephrin B2 mediated functions have been previously described by the present inventors (see, e.g., U.S. Pat. Nos. 7,381,410; 7,862,816; 7,977,463; 8,063,183; 8,273,858; 8,975,377; 8,981,062; 9,533,026; each hereby incorporated by reference in their entirety for all purposes).

The present inventors have identified the EphB4 receptor as a potential novel target in PC. Genetic validation studies with conditional deletion of EphB4 in the context of Pten deletion in prostate epithelium abolished tumor formation, and abrogated PI3K pathway induction. Additionally, a therapeutic agent, soluble EphB4 (sEphB4), prevented tumor formation and induced tumor regression in established pre-castration and post-castration tumors. Surprisingly, AR levels also declined with therapy. PI3K isoform analysis showed downregulation of only PI3K p110 beta which directly regulates AR levels, such that AR decline was rescued with ectopic expression of PI3K beta.

### INCORPORATION BY REFERENCE

Patent documents U.S. Pat. Nos. 7,381,410; 7,862,816; 7,977,463; 8,063,183; 8,273,858; 8,975,377; 8,981,062; 9,533,026; and all references disclosed herein are hereby incorporated by reference in their entirety for all purposes.

### DISCLOSURE OF THE INVENTION

Provided herein are novel methods and compositions for treating prostate cancer in a subject. In one aspect, the present invention relates to use of a polypeptide agent that inhibits EphB4 or Ephrin B2 mediated functions in the preparation of a medicament for use in treating prostate cancer (PC). More specifically, for use in treating PTEN deficient PC or PC that is refractory to treatment using androgen receptor (AR) targeted therapy.

In various embodiments, the polypeptide agent that inhibits EphB4 or Ephrin B2 mediated functions is a monomeric ligand binding portion of the EphB4 protein or Ephrin B2 protein, or an antibody that binds to and affects EphB4 or Ephrin B2. In various embodiments, the polypeptide agent is a soluble EphB4 (sEphB4) polypeptide that binds specifically to an Ephrin B2 polypeptide and comprises an amino acid sequence of an extracellular domain of an EphB4 protein. In various embodiments, the sEphB4 polypeptide comprises a globular domain of an EphB4 protein.

In various embodiments, the sEphB4 polypeptide comprises a sequence selected from the group consisting of a sequence that is at least 90% identical to residues 1-522, at least 90% identical to residues 1-412, and at least 90% identical to residues 1-312 of the amino acid sequence of SEQ ID NO: 1. In various embodiments, the sEphB4 polypeptide may comprise a sequence encompassing the globular (G) domain (amino acids 29-197 of SEQ ID NO; 1), and optionally additional domains, such as the cysteine-rich domain (amino acids 239-321 of SEQ ID NO: 1), the first fibronectin type 3 domain (amino acids 324-429 of SEQ ID NO: 1) and the second fibronectin type 3 domain (amino acids 434-526 of SEQ ID NO: 1). In various embodiments, the sEphB4 polypeptide will comprise amino acids 1-537 of SEQ ID NO: 1. In various embodiments, the sEphB4 polypeptide will comprise amino acids 1-427 of SEQ ID NO: 1. In various embodiments, the sEphB4 polypeptide will comprise amino acids 1-326 of SEQ ID NO: 1. In various embodiments, the sEphB4 polypeptide will comprise amino acids 1-197, 29-197, 1-312, 29-132, 1-321, 29-321, 1-326, 29-326, 1-412, 29-412, 1-427, 29-427, 1-429, 29-429, 1-526, 29-526, 1-537 and 29-537 of SEQ ID NO: 1. In various embodiments, the sEphB4 polypeptide will comprise amino acids 16-197, 16-312, 16-321, 16-326, 16-412, 16-427, 16-429, 16-526, and 16-537 of SEQ ID NO: 1.

In various embodiments, a soluble polypeptide may be prepared in a multimeric form, by, for example, expressing as an Fc fusion protein or fusion with another multimerization domain.

In various embodiments, the sEphB4 polypeptide will further comprise an additional component that confers increased serum half-life while still retaining Ephrin B2 binding activity. In various embodiments, the sEphB4 polypeptides are monomeric and are covalently linked to one or more polyoxyaklylene groups (e.g., polyethylene, polypropylene). In various embodiments, the sEphB4 polypeptide is covalently linked to a polyethylene glycol (PEG) group(s) (hereinafter “sEphB4-PEG”).

In various embodiments, the sEphB4 polypeptide is stably associated with a second stabilizing polypeptide that confers improved half-life without substantially diminishing Ephrin B2 binding. In various embodiments, the stabilizing polypeptide is immunocompatible with human patients (or animal patients, where veterinary uses are contemplated) and will have little or no significant biological activity. In various embodiments, the sEphB4 polypeptide is associated covalently or non-covalently with an albumin selected from the group consisting of a human serum albumin (HSA) (hereinafter “sEphB4-HSA”) and bovine serum albumin (BSA) (hereinafter “sEphB4-BSA”). In various embodiments, the sEphB4-HSA comprises residues 16-197 of SEQ ID NO: 1 directly fused to residues 25-609 of SEQ ID NO: 2. In various embodiments, the sEphB4-HSA comprises residues 16-312 of SEQ ID NO: 1 directly fused to residues 25-609 of SEQ ID NO: 2. In various embodiments, the sEphB4-HSA comprises residues 16-321 of SEQ ID NO: 1 directly fused to residues 25-609 of SEQ ID NO: 2. In various embodiments, the sEphB4-HSA comprises residues 16-326 of SEQ ID NO: 1 directly fused to residues 25-609 of SEQ ID NO: 2. In various embodiments, the sEphB4-HSA comprises residues 16-412 of SEQ ID NO: 1 directly fused to residues 25-609 of SEQ ID NO: 2. In various embodiments, the sEphB4-HSA comprises residues 16-427 of SEQ ID NO: 1 directly fused to residues 25-609 of SEQ ID NO: 2. In various embodiments, the sEphB4-HSA comprises residues 16-429 of SEQ ID NO: 1 directly fused to residues 25-609 of SEQ ID NO: 2. In various embodiments, the sEphB4-HSA comprises residues 16-526 of SEQ ID NO: 1 directly fused to residues 25-609 of SEQ ID NO: 2. In various embodiments, the sEphB4-HSA comprises residues 16-537 of SEQ ID NO: 1 directly fused to residues 25-609 of SEQ ID NO: 2.

In another aspect, the present invention relates to the use of a polypeptide agent that inhibits EphB4 or Ephrin B2 mediated functions in the preparation of a medicament for use in combination therapy for treating a cancer in a subject. In various embodiments, the combination therapy relates to methods of treating prostate cancer in a subject, comprising administering to the subject a) a therapeutically effective amount of an sEphB4-HSA polypeptide, and b) a therapeutically effective amount of a second anti-cancer therapy. The combination therapy may be synergistic. The combination therapy may increase the therapeutic index of the anti-cancer therapy.

In various embodiments, the second anti-cancer therapy is selected from the group consisting of androgen deprivation therapy (ADT), AR targeted therapy, hormone deprivation therapy, immunotherapy, chemotherapy, targeted treatment using depleting antibodies to specific tumor antigens, targeted treatment using agonistic, antagonistic, or blocking antibodies to co-stimulatory or co-inhibitory molecules (immune checkpoints), targeted treatment with an immunoconjugate, ADC, or fusion molecule comprising depleting antibodies to specific tumor antigens and a cytotoxic agent, small molecule kinase inhibitor targeted therapy, surgery, radiation therapy, treatment using DHT blockers, and stem cell transplantation. In various embodiments, the second anti-cancer therapy comprises administration of an antibody that specifically binds an immune-checkpoint protein antigen from the list including, but not limited to, CD276, CD272, CD152, CD223, CD279, CD274, TIM-3 and B7-H4; or any immune-checkpoint protein antigen antibody taught in the art.

In various embodiments, the subject has resistant or refractory prostate cancer. In various embodiments, the cancer is refractory to chemotherapy. In various embodiments, the cancer is refractory to androgen deprivation therapy (ADT). In various embodiments, the cancer is refractory to AR targeted therapy. In various embodiments, the cancer is refractory to hormone deprivation therapy. In various embodiments, the cancer is refractory to immunotherapy treatment. In various embodiments, the cancer is refractory to treatment using depleting antibodies to specific tumor antigens. In various embodiments, the cancer is refractory to treatment using agonistic, antagonistic, or blocking antibodies to co-stimulatory or co-inhibitory molecules (immune checkpoints). In various embodiments, the cancer is refractory to targeted treatment with an immunoconjugate, antibody-drug conjugate (ADC), or fusion molecule comprising a depleting antibody to specific tumor antigens tumor antigen and a cytotoxic agent. In various embodiments, the cancer is refractory to targeted treatment with a small molecule kinase inhibitor. In various embodiments, the cancer is refractory to treatment using surgery. In various embodiments, the cancer is refractory to treatment using stem cell transplantation. In various embodiments, the cancer is refractory to treatment using radiation. In various embodiments, the cancer is refractory to treatment using DHT blockers. In various embodiments, the cancer is refractory to combination therapy involving, for example, two or more of: androgen deprivation therapy, AR targeted therapy, hormone deprivation therapy, immunotherapy treatment, treatment with a chemotherapeutic agent, treatment with a tumor antigen-specific, depleting antibody, treatment with a immunoconjugate, ADC, or fusion molecule comprising a tumor antigen-specific, depleting antibody and a cytotoxic agent, targeted treatment with a small molecule kinase inhibitor, treatment using surgery, treatment using stem cell transplantation, treatment using DHT blockers and treatment using radiation.

In various embodiments, the subject previously responded to treatment with an anti-cancer therapy, but, upon cessation of therapy, suffered relapse (hereinafter “a recurrent proliferative disease”).

### BRIEF DESCRIPTION OF THE DRAWINGS

FIG. 1. Induction of EphrinB2-EphB4 in prostate cancers. (A) EphB/EphrinB family members gene expressions were performed in 492 prostate cancer patients with somatic mutation based on The Cancer Genome Atlas (TCGA). (B) Hematoxylin and eosin stain (H&E), and immunohistochemistry (IHC) stain of EphrinB2 in 148 human prostate cancer tissue array (Biomax Inc. PR1921b). EphrinB2 is highly expressed in prostate cancer but not normal prostate tissue. Top bar indicates the Gleason scores of prostate cancer. Upper two panels indicate lower magnifications (200×); lower two panels indicate higher magnifications (400×). The table shows EphrinB2 expression in Gleason score 6 and 7 above which has no significant changes. (C) The growth of primary prostate cancers in PTEN-null mice were followed noninvasively by bioluminescence imaging (BLI). Luciferase signal expression was increased over time from age of 4-month to 7-month. Colors indicates of the intensity of luciferase signal. Blue denotes weak signal; Green and yellow denote intermediate signal; Red denotes strong signal. (D) H&E and IHC of EphB4, PTEN, phosphorylated AKT (pAKT), phosphorylated S6 (pS6) protein level in of wild type (WT) mice prostate glands versus Cre-PTEN^{-/-}-Luciferase (CPPL) mice prostate glands. (E) Western blot of EphB4, EphB2, EphB3 and Pten protein level in one wild type (WT) and three CPPL mice. Glyceraldehyde 3-phosphate dehydrogenase (GAPDH) was probed to ensure equal loading. (F) Quantitative RT-PCR of EphB4, EphB1, EphB2, and EphB3 RNA level were measured in wild type (blue) and CPPL mice (red). Gene expression was normalized against β-actin. Error bars represent standard deviation (s.d.). These experiments were repeated at least two times and similar results were obtained. P-value was calculated using two-tailed, unpaired Student's t-test. **P<0.002.

FIG. 2. EphB4 genetic mouse model. (A) Schematic representation of the inducible EphB4 conditional knockout (EphB4 CKO) mice with floxed allele. Numbers indicate the exons 1-4. Bold black arrows indicate two loxp sites flanked around exon 2 and 3. P1 and P2 indicate PCR primer sites for genotyping. (B) Genotyping PCR confirmed wild type, heterozygous and homozygous floxed Pten and EphB4. Left panel: wild type EphB4 (WT, EphB4^{wild-type/wild-type}) has one 189 bp band, heterozygous EphB4 (F/+, EphB4^{floxed/wild-type}) has two bands with sizes of 189 bp and 282 bp and homozygous EphB4 (F/F, EphB4^{floxed/floxed}) has one 282 bp band. Right panel: wild type Pten (WT, Pten^{wild-type/wild-type}) has one 321 bp band; heterozygous Pten (F/+, Pten^{floxed/wild-type}) has two bands with sizes of 321 bp and 493 bp and homozygous Pten (F/F, Pten^{floxed/floxed}) has one 493 bp band. bp denotes base pair. (C) Quantitative RT-PCR of EphB4, EphB1, EphB2, and EphB3 RNA level were measured in wild type mice (both EphB4 and Pten are wild type, blue), CPPL mice (CPPL mice with wild type of EphB4, red), EphB4 hetero-CPPL mice (CPPL mice with heterozygous deletion of EphB4, green) and EphB4homo-CPPL mice (CPPL mice with homozygous deletion of EphB4, purple) prostate tissues. Gene expression was normalized against β-actin. Error bars represent standard deviation (s.d.). These experiments were repeated at least three times and similar results were obtained. P-value was calculated using two-tailed, unpaired Student's t-test. *P<0.05; **P<0.002.

FIG. 3. Knockout of EphB4 inhibits PTEN deletion-induced prostate cancer progression. (A) Luciferase expression of live mice imaging over the time including age 3-month, 5-month and 7-month. EphB4^{+/+};CPPL denotes CPPL mice with wild type of EphB4. EpHB4^{F/F};CPPL denotes CPPL mice with homozygous type of EphB4. Left panel represented of 3 out of 14 EphB4^{+/+};CPPL. Right panel represented of 3 out of 29 EpHB4^{F/F};CPPL. Color bars indicate the intensity of luciferase signal. Blue denotes weak signal; Green and yellow denote intermediate signal; Red denotes strong signal. (B) Quantitative measurement of bioluminescence imaging (BLI) signal intensity of total 14 EphB4^{+/+}*;CPPL mice and 29 EpHB4^{F/F};CPPL mice. Blue: age of 3-month. Red: age of 5-month. Green: age of 7-month. Intensity of BLI was labelled on the top of each bar. Error bars represent standard deviation (s.d.). (C) Prostate gland was dissected from in EphB4 wild type PTEN null mice (EphB4^{+/+}*;CPPL), EphB4 heterozygote PTEN null mice (EphB4^{F/+;} CPPL) and EphB4 homozygote PTEN null mice (EphB4^{F}/F; CPPL). Red dash lines denote the tumor areas. (D) Western blot of EphB4 was performed from tumors harvested from the prostate gland of mice. 3-actin was probed to ensure equal loading. These experiments were repeated at least three times and similar results were obtained. (E) H&E stain of prostate glands from WT, EphB4^{+/+};CPPL and EpHB4^{F/F};CPPL mice in different magnifications 100×, 200× and 400×, respectively. (F) IHC stain of Ki67 in prostate tissues. Signal intensities showed on right panel were quantified based on positive staining pixel dots using ImageJ (NIH) software. All error bars represent s.d. (G) IHC stain of EphB4, PTEN, pAKT and pS6 in prostate tissues. WT: wild type both EphB4 and Pten; EphB4^{+/+};CPPL: EphB4 wild type PTEN null mice; EpHB4^{F/F};CPPL: EphB4 homozygote PTEN null mice. All these experiments were repeated at least three times and similar results were obtained.

FIG. 4. Inhibition of EphB4 signaling by sEphB4 prevents PTEN deletion-induced prostate cancer progression. (A) Luciferase expression of live mice imaging of age 4-mo, 5-mo, 6-mo and 7-mo corresponding to the length of treatment 0-mo, 1-mo, 2-mo and 3-mo, respectively. Left panel: CPPL treated with phosphate-buffered saline (PBS) which serves as control. Right panel: CPPL treated with soluble EphB4-Albumin (sEphB4-Alb). Three mice from each group were represented. Color bars indicate the intensity of luciferase signal. (B) Quantitative measurement of bioluminescence imaging (BLI) signal intensity of total 8 CPPL;PBS-treated mice and 15 CPPL;sEphB4-treated mice. Intensity of BLI was labelled on the top of each bar. (C) Luciferase expression of live mice imaging of age 7-mo, 8-mo, 9-mo and 10-mo corresponding to the length of sEphB4-Albumin treatment 0-mo, 1-mo, 2-mo and 3-mo, respectively. Three mice were represented. Color bars indicate the intensity of luciferase signal. (D) Quantitative measurement of bioluminescence imaging (BLI) signal intensity of total 8 CPPL;sEphB4-treated mice. Blue: 0-month treatment. Red: 1-month treatment. Green: 2-month treatment. Purple: 3-month treatment. Intensity of BLI was labelled on the top of each bar. Error bars represent standard deviation (s.d.). (E) TUNEL immunoassay (upper panel) of prostate tissues from PBS-treated versus sEphB4-Albumin treated CPPL mice and lower panel is H&E stain of the serial slides from the same tissue. (F) IHC stain of EphB4, pAKT and pS6 in wild type (WT), PBS treated CPPL (CPPL;PBS) and sEphB4-Albumin treated CPPL (CPPL;sEphB4-Alb) prostate tissues. All these experiments were repeated at least three times and similar results were obtained.

FIG. 5. Inhibition of EphB4 signaling by sEphB4 inhibits androgen resistant prostate cancer progression. (A) Luciferase expression of live mice imaging of age 4-mo, 5.5-mo, 8.5-mo, 10-mo and 14.5-mo corresponding to post-castration 0-mo, 1.5-mo, 4.5-mo, 6-mo and 10.5-mo, respectively. Mice was started treatment with either PBS or sEphb4-Albumin from age of 8.5-month in both groups. Left panel: CPPL treated with phosphate-buffered saline (PBS). Right panel: CPPL treated with soluble EphB4-Albumin (sEphB4-Alb). Three mice from each group were represented. (B) Color bars indicate the intensity of luciferase signal. Quantitative measurement of bioluminescence imaging (BLI) signal intensity of total 8 CPPL;sEphB4-treated mice. Intensity of BLI was labelled on the top of each bar. Error bars represent standard deviation (s.d.).

FIG. 6. EphB4 functions in PI3K pathway in vitro. (A) Western blot of EphB4, P110α, P110β, P110γ, phosphorylated AKT (pAKT), AKT, phosphorylated S6 (PS6), S6, phosphorylated P38 (pP38), P38 in two human prostate cancer cell lines, C4-2B and PC3. EphB4 siRNA specifically down-regulated PI3K downstream markers (pAKT, pS6) and P110 subunit β, but not p110α and γ in both PC3 and C4-2B cell lines. 3-actin was probed to ensure equal loading. Experiment was performed in triplicate. (B) Immunofluorescent (IF) stain of phosphatidylinositol (3,4,5) triphosphate (PIP3). DAPI denotes nuclear staining. Merge of PIP3 and DAPI. PIP3 was also diminished with EphB4 siRNA knockdown. (C) Western blot of EphB4 and it confirmed EphB4 knockdown with EphB4 siRNA in all test samples. (D) Western blot of AKT and pAKT. Both overexpression of wild-type AKT (wt-AKT) and constitutionally active forms of AKT (ΔAKT) rescued AKT and pAKT level from EphB4siRNA. Vector: empty vector as control. 3-actin was probed to ensure equal loading. (E) Cell viability (MTT) assay confirmed overexpression of either wt-AKT or ΔAKT rescued the cell death caused by siEphB4 compared to control siRNA but not empty vector. Cell viability percentage was labelled on the top of each bar. Experiment was performed in triplicate.

FIG. 7. PI3K pathway regulates AR via P110β isoform. (A) Western blot showed the expression level of P85, P110α, P110β, P110γ, and P110δ in different cancer cell lines 22RV1, C4-2B, PC3, K562, and RAJI. (B) Western blot of S6, pS6, EphB4 and AR in 22RV1 prostate cell line after treating with inhibitors of P110α (BYL719), P1103 (GSK2636771), P110γ (IPI-549), and P110δ (GSK2269557) at various concertation. Inhibition of α and β isoforms significantly reduced pS6 levels. Only inhibition of P110β reduced EphB4 and AR while inhibitors of P110α/γ/δ had no effect on either proteins. Bottom panel demonstrates the quantitative analysis of relative pS6/S6 ratio, EphB4 and AR protein level was made using ImageJ (NIH). β-actin was probed to ensure equal loading. Experiment was performed in triplicate.

FIG. 8. EphB4 regulates AR via P110β isoform. (A) Western blot of EphB4 and AR in 22RV1 and C4-2B cells. AR was significantly downregulated when knockdown EphB4 using EphB4siRNA (B4si) compared to control siRNA (ctrlsi) and no siRNA (mock). 3-actin was probed to ensure equal loading. (B) IHC stain with anti-AR antibody (brown color) in mouse prostate tissues from wild type (WT), PBS treated CPPL (CPPL;PBS), EphB4 knockout CPPL (CPPL;EphB4F/F), and soluble EphB4-Alb treated CPPL (CPPL;sEphB4-Alb) at 100×, 400×, 1000× magnification. Dash rectangle denotes the corresponding picture showed in higher magnification. (C) mRNA level of AR in EphB4-knockdown 22RV1 and C4-2B cells by Quantitative RT-PCR using EphB4siRNA compared to control siRNA and no siRNA (mock). Left panel showed the quantitative analysis of relative EphB4 and AR level which were significantly reduced in siEphB4 treated C4-2B and 22RV1 cell lines. Experiment was performed in triplicate. P-value was calculated using two-tailed, unpaired Student's t-test. NS, not significant; *P<0.05; (D) AR rescue experiments. Western blot of AR, EphB4, AKT, p110α, p110β, and p110γ in 22RV1 cells with co-transfection of EphB4 siRNA and plasmids of EphB4, p110α, p110β, and p110γ, respectively. The quantitative analysis of relative AR level was made using ImageJ (NIH). In this experiment, the reduced AR level caused by EphB4 siRNA can be rescued by overexpression of Akt and p1103 but not p110α and p110γ. Interestingly, overexpression of p110α and p110γ can further inhibit AR level which is consistent with other studies.

FIG. 9. sEphB4 decreased the PSA level in a CRPC patient. H&E and IHC stain of EphrinB2 and CD31 of prostate tumor biopsy. Right lower graph showed PSA level changes after sEphB4-Alb treatment over weeks.

FIG. 10. EphB4 CKO1 (−/−); CMV-Cre (+) mice phenotype. Mice embryonic dissection at day E9.5. One EphB4 CKO1 (wt/wt) embryo and three EphB4 CWKO1 (−/−) embryos with embryonic lethal.

FIG. 11. The intensity of BLI of CPPL mice versus Eph4-knockout CPPL mice. Chart summarized the BLI intensity based on age. Each dot denotes each mouse. Red line showed the average BLI intensity of all mice at certain age. Left panel: EphB4 wild type CPPL mice (CPPL only). Right panel: Eph4-knockout CPPL mice (EphB4(F/F);CPPL). Bottom of each panel showed the alive mice numbers corresponding to the age group.

FIG. 12. The intensity of BLI of PBS treated CPPL mice versus sEph4-Alb treated CPPL mice. Chart summarized the BLI intensity based on age. Each dot denotes each mouse. Red line showed the average BLI intensity of all mice at certain age. Left panel: PBS treated CPPL mice. Right panel: sEph4-Alb treated CPPL mice. Bottom of each panel showed the alive mice numbers corresponding to the age group.

FIG. 13. The intensity of BLI of PBS treated sEph4-Alb treated CPPL mice in regression experiment. Chart summarized the BLI intensity based on age. Each dot denotes each mouse. Red line showed the average BLI intensity of all mice at certain age. Bottom of panel showed the alive mice numbers corresponding to the age group.

FIG. 14. AR level of knockdown of EphB4 in prostate cancer cell lines. RT-PCT curves indicate the gene level changes of AR, EphB4, GADPH with EphB4 siRNA versus control siRNA in C4-2B (Left) and 22RV1 (Right) cell lines.

### MODE(S) FOR CARRYING OUT THE INVENTION

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those commonly used and well known in the art. The methods and techniques of the present invention are generally performed according to conventional methods well known in the art and as described in various general and more specific references that are cited and discussed throughout the present specification unless otherwise indicated. See, e.g., Green and Sambrook, Molecular Cloning: A Laboratory Manual, 4th ed., Cold Spring Harbor Laboratory Press, Cold Spring Harbor, N.Y. (2012), incorporated herein by reference. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those commonly used and well known in the art. Standard techniques are used for chemical syntheses, chemical analyses, pharmaceutical preparation, formulation, and delivery, and treatment of subjects.

As used herein, a “proliferative disease” includes tumor disease (including benign or cancerous) and/or any metastases. A proliferative disease may include hyperproliferative conditions such as hyperplasias, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. In various embodiments, the proliferative disease is cancer. In various embodiments, the proliferative disease is a non-cancerous disease. In various embodiments, the proliferative disease is a benign or malignant tumor.

The term “tumor,” as used herein, refers to all neoplastic cell growth and proliferation, whether malignant or benign, and all pre-cancerous and cancerous cells and tissues.

The term “primary tumor” refers to all neoplastic cell growth and proliferation, whether malignant or benign, and all pre-cancerous and cancerous cells and tissues located at the anatomical site where the autonomous, unregulated growth of the cells initiated, for example the organ of the original cancerous tumor. Primary tumors do not include metastases.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor (e.g., the organ containing the primary tumor). Metastasis can also be defined as several steps of a process, such as the departure of cancer cells from an original tumor site (e.g., primary tumor site) and migration and/or invasion of cancer cells to other parts of the body.

“Resistant or refractory cancer” refers to tumor cells or cancer that do not respond to previous anti-cancer therapy including, e.g., androgen deprivation therapy (ADT), hormone deprivation therapy, chemotherapy, surgery, radiation therapy, stem cell transplantation, and immunotherapy. Tumor cells can be resistant or refractory at the beginning of treatment, or they may become resistant or refractory during treatment. Refractory tumor cells include tumors that do not respond at the onset of treatment or respond initially for a short period but fail to respond to treatment. Refractory tumor cells also include tumors that respond to treatment with anticancer therapy but fail to respond to subsequent rounds of therapies. For purposes of this invention, refractory tumor cells also encompass tumors that appear to be inhibited by treatment with anticancer therapy but recur up to five years, sometimes up to ten years or longer after treatment is discontinued. The anticancer therapy can employ chemotherapeutic agents alone, radiation alone, targeted therapy alone, surgery alone, or combinations thereof. For ease of description and not limitation, it will be understood that the refractory tumor cells are interchangeable with resistant tumor cells.

As used herein, “treatment” (and grammatical variations thereof such as “treat” or “treating”) refers to clinical intervention in an attempt to alter the natural course of a disease in the individual being treated and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

The term “effective amount” or “therapeutically effective amount” as used herein refers to an amount of a compound or composition sufficient to treat a specified disorder, condition or disease such as ameliorate, palliate, lessen, and/or delay one or more of its symptoms. In reference to NHL and other cancers or other unwanted cell proliferation, an effective amount comprises an amount sufficient to: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. An effective amount can be administered in one or more administrations.

“Adjuvant setting” refers to a clinical setting in which an subject has had a history of a proliferative disease, particularly cancer, and generally (but not necessarily) been responsive to therapy, which includes, but is not limited to, surgery (such as surgical resection), radiotherapy, and chemotherapy. However, because of their history of the proliferative disease (such as cancer), these subjects are considered at risk of development of the disease. Treatment or administration in the “adjuvant setting” refers to a subsequent mode of treatment. The degree of risk (i.e., when an subject in the adjuvant setting is considered as “high risk” or “low risk”) depends upon several factors, most usually the extent of disease when first treated.

The phrase “synergistic effect” refers to the effect achieved when the active ingredients used together is greater than the sum of the effects that results from using the active ingredients separately. The terms “synergy”, “synergism”, “synergistic”, “combined synergistic amount”, and “synergistic therapeutic effect” are used herein interchangeably.

The phrase “administering” or “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic regimen, and/or prescribing particular agent(s)/compounds for a subject. Such prescribing can include, for example, drafting a prescription form, annotating a medical record, and the like. Where administration is described herein, “causing to be administered” is also contemplated.

The terms “patient,” “individual,” and “subject” may be used interchangeably and refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In various embodiments, the patient can be a human (e.g., adult male, adult female, adolescent male, adolescent female, male child, female child) under the care of a physician or other health worker in a hospital, psychiatric care facility, as an outpatient, or other clinical context. In various embodiments, the patient may be an immunocompromised patient or a patient with a weakened immune system including, but not limited to patients having primary immune deficiency, AIDS; cancer and transplant patients who are taking certain immunosuppressive drugs; and those with inherited diseases that affect the immune system (e.g., congenital agammaglobulinemia, congenital IgA deficiency). In various embodiments, the patient has an immunogenic cancer, including, but not limited to bladder cancer, lung cancer, melanoma, and other cancers reported to have a high rate of mutations (Lawrence et al., Nature, 499(7457): 214-218, 2013).

As used herein, the terms “co-administration”, “co-administered” and “in combination with”, referring to the fusion molecules of the invention and one or more other therapeutic agents, is intended to mean, and does refer to and include the following: simultaneous administration of such combination of fusion molecules of the invention and therapeutic agent(s) to an subject in need of treatment, when such components are formulated together into a single dosage form which releases said components at substantially the same time to said subject; substantially simultaneous administration of such combination of fusion molecules of the invention and therapeutic agent(s) to an subject in need of treatment, when such components are formulated apart from each other into separate dosage forms which are taken at substantially the same time by said subject, whereupon said components are released at substantially the same time to said subject; sequential administration of such combination of fusion molecules of the invention and therapeutic agent(s) to an subject in need of treatment, when such components are formulated apart from each other into separate dosage forms which are taken at consecutive times by said subject with a significant time interval between each administration, whereupon said components are released at substantially different times to said subject; and sequential administration of such combination of fusion molecules of the invention and therapeutic agent(s) to an subject in need of treatment, when such components are formulated together into a single dosage form which releases said components in a controlled manner whereupon they are concurrently, consecutively, and/or overlappingly released at the same and/or different times to said subject, where each part may be administered by either the same or a different route.

The terms “polypeptide”, “peptide” and “protein” are used interchangeably herein to refer to a polymer of amino acid residues. In certain embodiments, “peptides”, “polypeptides”, and “proteins” are chains of amino acids whose alpha carbons are linked through peptide bonds. The terminal amino acid at one end of the chain (amino terminal) therefore has a free amino group, while the terminal amino acid at the other end of the chain (carboxy terminal) has a free carboxyl group. As used herein, the term “amino terminus” (abbreviated N-terminus) refers to the free α-amino group on an amino acid at the amino terminal of a peptide or to the α-amino group (imino group when participating in a peptide bond) of an amino acid at any other location within the peptide. Similarly, the term “carboxy terminus” refers to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether as opposed to an amide bond.

The term “recombinant polypeptide”, as used herein, is intended to include all polypeptides, including fusion molecules that are prepared, expressed, created, derived from, or isolated by recombinant means, such as polypeptides expressed using a recombinant expression vector transfected into a host cell.

Polypeptides of the disclosure include polypeptides that have been modified in any way and for any reason, for example, to: (1) reduce susceptibility to proteolysis, (2) reduce susceptibility to oxidation, (3) alter binding affinity for forming protein complexes, (4) alter binding affinities, and (5) confer or modify other physicochemical or functional properties. For example, single or multiple amino acid substitutions (e.g., conservative amino acid substitutions) may be made in the naturally occurring sequence (e.g., in the portion of the polypeptide outside the domain(s) forming intermolecular contacts). A “conservative amino acid substitution” refers to the substitution in a polypeptide of an amino acid with a functionally similar amino acid. The following six groups each contain amino acids that are conservative substitutions for one another:

Alanine (A), Serine (S), and Threonine (T)

Aspartic acid (D) and Glutamic acid (E)

Asparagine (N) and Glutamine (Q)

Arginine (R) and Lysine (K)

Isoleucine (I), Leucine (L), Methionine (M), and Valine (V)

Phenylalanine (F), Tyrosine (Y), and Tryptophan (W)

The term “polypeptide fragment” and “truncated polypeptide” as used herein refers to a polypeptide that has an amino-terminal and/or carboxy-terminal deletion as compared to a corresponding full-length protein. In certain embodiments, fragments can be, e.g., at least 5, at least 10, at least 25, at least 50, at least 100, at least 150, at least 200, at least 250, at least 300, at least 350, at least 400, at least 450, at least 500, at least 600, at least 700, at least 800, at least 900 or at least 1000 amino acids in length. In certain embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

The terms “polypeptide variant” and “polypeptide mutant” as used herein refers to a polypeptide that comprises an amino acid sequence wherein one or more amino acid residues are inserted into, deleted from and/or substituted into the amino acid sequence relative to another polypeptide sequence. In certain embodiments, the number of amino acid residues to be inserted, deleted, or substituted can be, e.g., at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Variants of the present disclosure include fusion proteins.

The term “soluble polypeptide” as used herein merely indicates that the polypeptide does not contain a transmembrane domain or a portion of a transmembrane domain sufficient to compromise the solubility of the polypeptide in a physiological salt solution.

“Pharmaceutical composition” refers to a composition suitable for pharmaceutical use in an animal. A pharmaceutical composition comprises a pharmacologically effective amount of an active agent and a pharmaceutically acceptable carrier. “Pharmacologically effective amount” refers to that amount of an agent effective to produce the intended pharmacological result. “Pharmaceutically acceptable carrier” refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” is a salt that can be formulated into a compound for pharmaceutical use including, e.g., metal salts (sodium, potassium, magnesium, calcium, etc.) and salts of ammonia or organic amines.

It is understood that aspect and embodiments of the invention described herein include “consisting” and/or “consisting essentially of” aspects and embodiments.

Reference to “about” a value or parameter herein includes (and describes) variations that are directed to that value or parameter per se. For example, description referring to “about X” includes description of “X”.

As used herein and in the appended claims, the singular forms “a,” “or,” and “the” include plural referents unless the context clearly dictates otherwise.

### MODE(S) FOR CARRYING OUT THE DISCLOSURE

The methods of the present disclosure include treating, reducing, or preventing primary tumor growth or formation of prostate cancer, or metastasis of prostate cancer by administering a polypeptide agent that inhibits EphB4 or Ephrin B2 mediated functions, either as monotherapy, or in combination with a second anti-cancer therapy.

### EphB4—Ephrin B2 Inhibitors

EphB4 is a member of the largest receptor tyrosine kinase family and the first of the 16 in vertebrates (EphA1) was cloned from the Erythropoietin Producing Hepatoma cell line. Eph receptor interating Ephrin(s) ligands are also membrane bound. EphA (EphA1-8,10) subgroup ligands (EphrinAl-5) localize to the cell surface via glycosyl-phosphatidylinositol GPI anchor, while B family ligands (ephrinB1-3) are transmembrane proteins (Gale et al., Neuron, 17(1):9-19, 1996). Being cell bound, Eph-ephrin interactions occur upon cell-cell contact. Dimers of Eph and Ephrin form high affinity heterotetrameric complexes which trigger bidirectional signal in receptor and ligand expressing cells (Dravis et al., Dev Biol, 271(2):272-290, 2004). Eph and ephrin regulate critical biological functions in cell localization, tissue boundary formation, and axon guidance. They are also required in proper positioning of precursor and mature cells in the intestinal crypt and villus cells (Jubb et al., Clin Cancer Res, 11(14):5181-5187, 2005), as well as urethral development in the genitourinary system (Peuckert et al., Kidney Int, 90(2):373-388, 2016).

The present inventor and others have previously shown induction of EphB4 in PC both at the level of gene and protein expression (Xia et al., Cancer Res, 65(11):4623-4632, 2005). Consistent with the potential role of EphB4 in PC, EphB4 has previously been shown to promote PC (Alazzouzi et al., Cancer Res, 65(22):10170-10173, 2005). The present inventors and others have shown previously that EphB4 is induced in prostate cancer by PI3K as well as β-catenin pathways (Batlle et al., Cell, 111(2):251-263, 2002; Kumar et al., Cancer Res, 69(9):3736-3745, 2009). EphB4 provides a survival advantage in tumor cells by further activating PI3K pathway. In contrast, EphB2, another member of the EphB receptor family functions as a tumor suppressor since loss of function mutations increase the risk for prostate cancer especially in African American men (Robbins et al. PLoS One, 6(5), el 949, 2011). The present inventors and others have also previously observed in colon cancer and bladder cancer that EphB2 expression declines during tumorigenesis, accompanied by the induction of EphB4 (Ozgur et al., Urol Oncol, 29(1):78-84, 2011; Stephenson et al., BMC Mol Biol, 2, 15, 2001).

EphB4 is not an oncogene by itself but may promote tumor initiation in the context of PTEN loss and/or PI3K activation. It has been previously shown that PI3K activation leads to EphB4 induction (R. Liu et al., BMC Cancer, 13:269, 2013). Secondly activation of EphB4 leads to induction of PI3K-AKT pathway activity. Since the PI3K pathway is induced in about 40% early in PC and nearly 100% in metastatic PC, mutations are observed frequently in PIKCA and PTEN in addition to growth factor mediated transcriptional addition in (Sarker et al., Clin Cancer Res, 15(15):4799-4805, 2009). Role of EphB4 in PC initiation is unknown to my knowledge.

It is also not known as to where EphB4 intersects in PI3K-AKT pathway. Since PI3K activates AKT, and constitutive activation of AKT rescued EphB4-deficient cells, it indicated that EphB4 intersects at or above AKT. EphB4 could thus engage at the level of PI3K itself. Class I PI3K has four catalytic isoforms p110 α, β, γ and δ, and each makes a dimer with a regulatory subunit to modulate the activity and subcellular localization of the complex. Since EphB4 knock down reduced only PI3K p110β and it is the form most prominent in PTEN deficient cells, it is likely to be the nodal point. Epithelial cells depend on the PI3K p110α isoform, but when PTEN is deleted, it signals through PI3K p110β for tumor progression.

Polypeptide agents that inhibit EphB4 or Ephrin B2 mediated functions have been previously described by the present inventors (see, e.g., U.S. Pat. Nos. 7,381,410; 7,862,816; 7,977,463; 8,063,183; 8,273,858; 8,975,377; 8,981,062; 9,533,026; each hereby incorporated by reference in their entirety for all purposes). In various embodiments of the present invention, the polypeptide agent that inhibits EphB4 or Ephrin B2 mediated functions is a monomeric ligand binding portion of the EphB4 protein or Ephrin B2 protein, or an antibody that binds to and affects EphB4 or Ephrin B2. In various embodiments, the polypeptide agent is a soluble EphB4 (sEphB4) polypeptide that binds specifically to an Ephrin B2 polypeptide and comprises an amino acid sequence of an extracellular domain of an EphB4 protein. In various embodiments, the sEphB4 polypeptide comprises a globular domain of an EphB4 protein.

In various embodiments, the sEphB4 polypeptide comprises a sequence selected from the group consisting of a sequence that is at least 90% identical to residues 1-522, at least 90% identical to residues 1-412, and at least 90% identical to residues 1-312 of the amino acid sequence of SEQ ID NO: 1. In various embodiments, the sEphB4 polypeptide may comprise a sequence encompassing the globular (G) domain (amino acids 29-197 of SEQ ID NO; 1), and optionally additional domains, such as the cysteine-rich domain (amino acids 239-321 of SEQ ID NO: 1), the first fibronectin type 3 domain (amino acids 324-429 of SEQ ID NO: 1) and the second fibronectin type 3 domain (amino acids 434-526 of SEQ ID NO: 1). In various embodiments, the sEphB4 polypeptide will comprise amino acids 1-537 of SEQ ID NO: 1. In various embodiments, the sEphB4 polypeptide will comprise amino acids 1-427 of SEQ ID NO: 1. In various embodiments, the sEphB4 polypeptide will comprise amino acids 1-326 of SEQ ID NO: 1. In various embodiments, the sEphB4 polypeptide will comprise amino acids 1-197, 29-197, 1-312, 29-132, 1-321, 29-321, 1-326, 29-326, 1-412, 29-412, 1-427, 29-427, 1-429, 29-429, 1-526, 29-526, 1-537 and 29-537 of SEQ ID NO: 1. In various embodiments, the sEphB4 polypeptide will comprise amino acids 16-197, 16-312, 16-321, 16-326, 16-412, 16-427, 16-429, 16-526 of SEQ ID NO: 1. In various embodiments, a sEphB4 polypeptide may be one that comprises an amino acid sequence at least 90%, and optionally 95% or 99% identical to any of the preceding amino acid sequences while retaining Ephrin B2 binding activity. In various embodiments, any variations in the amino acid sequence from the sequence shown in SEQ ID NO: 1 are conservative changes or deletions of no more than 1, 2, 3, 4 or 5 amino acids, particularly in a surface loop region.

In various embodiments, a soluble polypeptide may be prepared in a multimeric form, by, for example, expressing as an Fc fusion protein or fusion with another multimerization domain.

In various embodiments, the sEphB4 polypeptide will further comprise an additional component that confers increased serum half-life while still retaining Ephrin B2 binding activity. In various embodiments, the sEphB4 polypeptides are monomeric and are covalently linked to one or more polyoxyaklylene groups (e.g., polyethylene, polypropylene). In various embodiments, the sEphB4 polypeptide is covalently linked to a single polyethylene glycol (PEG) group (hereinafter “sEphB4-PEG”). In various embodiments, the sEphB4 polypeptide is covalently linked to two, three, or more PEG groups.

In various embodiments, the one or more PEG may have a molecular weight ranging from about 1 kDa to about 100 kDa, about 10 to about 60 kDa, and about 10 to about 40 kDa. The PEG group may be a linear PEG or a branched PEG. In various embodiments, the soluble, monomeric sEphB4 conjugate comprises an sEphB4 polypeptide covalently linked to one PEG group of from about 10 to about 40 kDa (monoPEGylated EphB4), or from about 15 to 30 kDa, preferably via an s-amino group of sEphB4 lysine or the N-terminal amino group. In various embodiments, the sEphB4 is randomly PEGylated at one amino group out of the group consisting of the s-amino groups of sEphB4 lysine and the N-terminal amino group.

In various embodiments, the sEphB4 polypeptide is stably associated with a second stabilizing polypeptide that confers improved half-life without substantially diminishing Ephrin B2 binding. In various embodiments, the stabilizing polypeptide is immunocompatible with human patients (or animal patients, where veterinary uses are contemplated) and will have little or no significant biological activity. In various embodiments, the sEphB4 polypeptide is associated covalently or non-covalently with an albumin selected from the group consisting of a human serum albumin (HSA) (hereinafter “sEphB4-HSA”) and bovine serum albumin (BSA) (hereinafter “sEphB4-BSA”). sEphB4-HSA is a fully human fusion protein composed of soluble EphB4 extracellular domain fused at the C-terminus with albumin upon expression as a single seamless protein of 123.3 kDa. sEphB4-HSA specifically binds to Ephrin B2. Preliminary studies of sEphB4-HSA in tumor models show increase in T and NK cell migration into tumor. This is accompanied by the induction of ICAM-1 in the tumor vessels. ICAM-1 is an integrin that promotes attachment of T and NK cells to the endothelium followed by transmigration of cells into the tumor. sEphB4-HSA also shows downregulation of PI3K signaling by blocking EphB-Ephrin B2 interaction in tumor cell and tumor vessels. Ephrin B2, a transmembrane protein is induced in tumor vessels. Ephrin B2 binds several members of EphB receptor tyrosine kinase family that are induced in tumor cells. Ephrin B2-EphB4 induces bidirectional signaling. sEphB4-HSA blocks the signaling and promote immune cell trafficking into the tumor and inhibit survival signal in tumor cells by downregulating PI3K pathway.

In various embodiments, the covalent attachment may be achieved by expression of the sEphB4 polypeptide as a co-translational fusion with human serum albumin. The albumin sequence may be fused at the N-terminus, the C-terminus or at a non-disruptive internal position in the sEphB4 polypeptide. Exposed loops of the sEphB4 would be appropriate positions for insertion of an albumin sequence. Albumin may also be post-translationally attached to the sEphB4 polypeptide by, for example, chemical cross-linking. In various embodiments, the sEphB4 polypeptide may also be stably associated with more than one albumin polypeptide.

In various embodiments, the sEphB4-HSA fusion inhibits the interaction between Ephrin B2 and EphB4, the clustering of Ephrin B2 or EphB4, the phosphorylation of Ephrin B2 or EphB4, or combinations thereof. In various embodiments, the sEphB4-HSA fusion has enhanced in vivo stability relative to the unmodified wildtype polypeptide.

In various embodiments, the sEphB4-HSA comprises residues 16-197 of SEQ ID NO: 1 directly fused to residues 25-609 of SEQ ID NO: 2. In various embodiments, the sEphB4-HSA comprises residues 16-312 of SEQ ID NO: 1 directly fused to residues 25-609 of SEQ ID NO: 2. In various embodiments, the sEphB4-HSA comprises residues 16-321 of SEQ ID NO: 1 directly fused to residues 25-609 of SEQ ID NO: 2. In various embodiments, the sEphB4-HSA comprises residues 16-326 of SEQ ID NO: 1 directly fused to residues 25-609 of SEQ ID NO: 2. In various embodiments, the sEphB4-HSA comprises residues 16-412 of SEQ ID NO: 1 directly fused to residues 25-609 of SEQ ID NO: 2. In various embodiments, the sEphB4-HSA comprises residues 16-427 of SEQ ID NO: 1 directly fused to residues 25-609 of SEQ ID NO: 2. In various embodiments, the sEphB4-HSA comprises residues 16-429 of SEQ ID NO: 1 directly fused to residues 25-609 of SEQ ID NO: 2. In various embodiments, the sEphB4-HSA comprises residues 16-526 of SEQ ID NO: 1 directly fused to residues 25-609 of SEQ ID NO: 2. In various embodiments, the sEphB4-HSA comprises residues 16-537 of SEQ ID NO: 1 directly fused to residues 25-609 of SEQ ID NO: 2.

### Prostate Cancer

Prostate cancer is the most common non-cutaneous malignancy in men and the second leading cause of death in men from cancer in the western world. Prostate cancer results from the uncontrolled growth of abnormal cells in the prostate gland. Once a prostate cancer tumor develops, androgens, such as testosterone, promote prostate cancer tumor growth. At its early stages, localized prostate cancer is often treated with local therapy including, for example, surgical removal of the prostate gland and radiotherapy. However, when local therapy fails to cure prostate cancer, as it does in up to a third of men, the disease progresses into incurable metastatic disease (i.e., disease in which the cancer has spread from one part of the body to other parts). As used herein, the term “prostate cancer” is used in the broadest sense and refers to all stages and all forms of cancer arising from the tissue of the prostate gland. The term “prostate cancer” encompasses any type of malignant (i.e. non-benign) tumor located in prostatic tissues, such as e.g. prostatic adenocarcinoma, prostatic sarcoma, undifferentiated prostate cancer, prostatic squamous cell carcinoma, prostatic ductal transitional carcinoma and prostatic intraepithelial neoplasia.

According to the tumor, node, metastasis (TNM) staging system of the American Joint Committee on Cancer (AJCC), AJCC Cancer Staging Manual (7th Ed., 2010), the various stages of prostate cancer are defined as follows: Tumor: T1: clinically in apparent tumor not palpable or visible by imaging, T1a: tumor incidental histological finding in 5% or less of tissue resected, T1b: tumor incidental histological finding in more than 5% of tissue resected, Tic: tumor identified by needle biopsy; T2: tumor confined within prostate, T2a: tumor involves one half of one lobe or less, T2b: tumor involves more than half of one lobe, but not both lobes, T2c: tumor involves both lobes; T3: tumor extends through the prostatic capsule, T3a: extracapsular extension (unilateral or bilateral), T3b: tumor invades seminal vesicle(s); T4: tumor is fixed or invades adjacent structures other than seminal vesicles (bladder neck, external sphincter, rectum, levator muscles, or pelvic wail). Generally, a clinical T (cT) stage is T1 or T2 and pathologic T (pT) stage is T2 or higher. Node: N0: no regional lymph node metastasis; N1: metastasis in regional lymph nodes. Metastasis: M0: no distant metastasis; M1: distant metastasis present.

PC is driven by AR signaling and first line androgen deprivation therapy remains the cornerstone of therapy in advanced disease, however, tumors do become resistant to deprivation while retaining AR function. For example, AR gene amplification can lead to higher sensitivity, while mutations in ligand binding domain can bind estrogen, progesterone, and glucocorticoids and activate AR signaling. AR mutation can also lead to constitutive activation independent of ligand binding. Ironically activation of PI3K pathway often reduces AR levels, and thus inhibition of PI3K pathway with Pan-PI3K inhibitors upregulates AR pathway activity. Similarly, AR inhibition activates PI3K pathway, in part by sustained pAKT due to reduction of PHLPP phosphatase (Carver et al., Cancer Cell, 19(5):575-586, 2011). Hence, these two pathways inversely regulate each other, and offer escape mechanisms for the inhibitors of one another. As described herein, the present inventor decided to evaluate whether sEphB4-HSA has an effect on AR itself, postulating a potential increase in AR as seen with PI3K inhibitors. Accordingly, AR in tumors from the sEphB4-HSA-treated mice was measured. Surprisingly, marked reduction in AR levels was observed. The present inventor also evaluated whether sEphB4-HSA may have PTEN deficiency related PI3Kβ inhibition, and if PI3P was responsible for AR decline. It has been suggested that PI3Kβ induces AR expression (Zhu et al., Oncogene, 27(33), 4569-4579, 2008) but not PI3Kα. PI3K isotype specific inhibitors showed that PI3Kβ is responsible for regulating AR levels. Reduction in EphB4 was also most prominent with PI3K p110β inhibition. Similarly, EphB4 inhibition lowered AR which was rescued by expressing p110β. Thus, EphB4 not only regulates PI3K but also controls AR level.

### Method of Treatment

In various embodiments, the present invention is directed to a method of treating prostate cancer in a subject in need of such treatment comprising administering to the human a therapeutically effective amount of a polypeptide agent that inhibits EphB4 or Ephrin B2 mediated functions.

In various embodiments, the present invention is directed to a method of treating prostate cancer in a subject in need of such treatment comprising a) administering to the human a therapeutically effective amount of a polypeptide agent that inhibits EphB4 or Ephrin B2 mediated functions and b) administering a therapeutically effective amount of a second anti-cancer therapy. The combination therapy may be synergistic. The combination therapy may increase the therapeutic index of the anti-cancer therapy.

In various embodiments, the second anti-cancer therapy is selected from the group consisting of androgen deprivation therapy (ADT), AR targeted therapy, hormone deprivation therapy, immunotherapy, chemotherapy, targeted treatment using depleting antibodies to specific tumor antigens, targeted treatment using agonistic, antagonistic, or blocking antibodies to co-stimulatory or co-inhibitory molecules (immune checkpoints), targeted treatment with an immunoconjugate, ADC, or fusion molecule comprising depleting antibodies to specific tumor antigens and a cytotoxic agent, small molecule kinase inhibitor targeted therapy, surgery, radiation therapy, treatment using DHT blockers, and stem cell transplantation. In various embodiments, the second anti-cancer therapy comprises administration of an antibody that specifically binds an immune-checkpoint protein antigen from the list including, but not limited to, CD276, CD272, CD152, CD223, CD279, CD274, TIM-3 and B7-H4; or any immune-checkpoint protein antigen antibody taught in the art.

Androgen deprivation therapy (“ADT”) or androgen suppression therapy is performed to reduce the testicular production of testosterone. ADT includes surgical castration (orchiectomy). As used herein, “androgen” or “androgen compound” refers to testosterone, dihydrotestosterone, androstenedione, dehydroepiandrosterone, androstenediol, androsterone, and the like. In various embodiments, “androgen” refers to testosterone or dihydrotestosterone. As used herein, “anti-androgen compound” refers to any compound that can lower androgen levels in the body. The anti-androgen compounds can be small molecules, peptides, or proteins. In various embodiments, the anti-androgen compound refers to a compound used for chemical orchiectomy. In various embodiments, the anti-androgen compound is a gonadotropin-releasing hormone (GnRH) antagonist. In various embodiments, the anti-androgen compound is a gonadotropin-releasing hormone (GnRH) agonist. In various embodiments, the anti-androgen compound is a luteinizing hormone-releasing hormone (LHRH) agonist. In various embodiments, the anti-androgen compound is a luteinizing hormone-releasing hormone (LHRH) antagonist. In various embodiments, the anti-androgen compound is abarelix, abiraterone, apalutamide, bicalutamide, degarelix, enzalutamide, flutamide, goserelin, leuprorelin (also known as leuprolide), nilutamide, ozarelix, or a combination of two or more thereof. In various embodiments the anti-androgen compound is abarelix. In various embodiments the anti-androgen compound is abiraterone. In various embodiments the anti-androgen compound is apalutamide. In various embodiments the anti-androgen compound is bicalutamide. In embodiments the anti-androgen compound is degarelix. In various embodiments the anti-androgen compound is enzalutamide. In various embodiments the anti-androgen compound is flutamide. In embodiments the anti-androgen compound is goserelin. In various embodiments the anti-androgen compound is leuprorelin. In various embodiments the anti-androgen compound is nilutamide. In various embodiments the anti-androgen compound is ozarelix. In various embodiments, the anti-androgen compound is in the form of a pharmaceutically acceptable salt. In various embodiments, the agent is an agent targeting the AR signaling pathway, including more effective antiandrogens, inhibitors of CYP17, an enzyme required for androgen synthesis, inhibitors of 5α-reductase, inhibitors of HSP90 which protects AR from degradation, inhibitors of histone deacetylases which is required for optimal AR mediated transcription, as well as inhibitors of tyrosine kinase inhibitors.

As used herein, anti-hormone therapy is a type of hormone deprivation therapy that suppresses selected hormones or their effects. Anti-hormone activity can be achieved by antagonizing hormone function (e.g. with a hormone analog or antagonist, or compositions blocking the binding/association between the hormone and its receptor, (e.g. hormone blocking compositions or blockades)) and/or by preventing or reducing their production. This can be done with drugs, radiation, and/or surgical approaches. The suppression of certain hormones can be beneficial to patients with cancers where certain hormones prompt or help the growth of a tumor. For example, androgen deprivation therapy, using reagents, such as a gonadotropin releasing hormone (GnRH) agonist to reduce endogenous androgen production resulting in low androgen level in body, can be used in treating prostate cancer.

As used herein, the term “immunotherapy” refers to cancer treatments which include, but are not limited to, treatment using depleting antibodies to specific tumor antigens; treatment using antibody-drug conjugates; treatment using agonistic, antagonistic, or blocking antibodies to co-stimulatory or co-inhibitory molecules (immune checkpoints) such as CTLA-4, PD-1, OX-40, CD137, GITR, LAG3, TIM-3, and VISTA; treatment using bispecific T cell engaging antibodies (BiTE®) such as blinatumomab: treatment involving administration of biological response modifiers such as IL-2, IL-12, IL-15, IL-21, GM-CSF, IFN-α, IFN-3 and IFN-γ; treatment using therapeutic vaccines such as sipuleucel-T; treatment using dendritic cell vaccines, or tumor antigen peptide vaccines; treatment using chimeric antigen receptor (CAR)-T cells; treatment using CAR-NK cells; treatment using tumor infiltrating lymphocytes (TILs); treatment using adoptively transferred anti-tumor T cells (ex vivo expanded and/or TCR transgenic); treatment using TALL-104 cells; and treatment using immunostimulatory agents such as Toll-like receptor (TLR) agonists CpG and imiquimod.

Immunotherapy using agonistic, antagonistic, or blocking antibodies to co-stimulatory or co-inhibitory molecules (immune checkpoints) has been an area of extensive research and clinical evaluation. Immune checkpoint proteins include CTLA-4, PD-1, LAG-3, and TIM-3 as well as several others (Pardoll D M., Nat Rev Cancer, 12:252-64, 2012; Sharpe et al., Nat Immunol, 8:239-45, 2007). Under normal physiological conditions, immune checkpoints are crucial for the maintenance of self-tolerance (that is, the prevention of autoimmunity) and protect tissues from damage when the immune system is responding to pathogenic infection. It is now also clear that tumors co-opt certain immune-checkpoint pathways as a major mechanism of immune resistance, particularly against T cells that are specific for tumor antigens (Pardoll D M., Nat Rev Cancer, 12:252-64, 2012). Accordingly, treatment utilizing antibodies to immune checkpoint molecules including, e.g., CTLA-4 (ipilimumab), PD-1 (nivolumab; pembrolizumab; pidilizumab) and PD-L1 (BMS-936559; MPLD3280A; MED14736; MSB0010718C)(see, e.g, Philips and Atkins, International Immunology, 27(1); 39-46, October 2014), and OX-40, CD137, GITR, LAG3, TIM-3, and VISTA (see, e.g., Sharon et al., Chin J Cancer, 33(9): 434-444, September 2014; Hodi et al., N Engl J Med, 2010; Topalian et al., N Engl J Med, 366:2443-54) are being evaluated as new, alternative immunotherapies to treat patients with proliferative diseases such as cancer, and in particular, patients with refractory and/or recurrent cancers. Despite the dramatic benefits and significant promise demonstrated by several of these immunotherapies, they remain limited by concerns over potential severe side effects and the fact that many tumors lack the targeted antigen and will therefore evade treatment. In general, about 20% of patients with various cancers respond to PD-1/PD-L1 antibodies or CTLA-4 antibodies. As such, there remains a critical unmet need for new and improved immunotherapies to treat patients with recurrent cancers and/or refractory cancers who do not respond to immune checkpoint therapy (ICT).

In various embodiments, the additional therapy comprises administration of an antibody that specifically binds an immune-checkpoint protein antigen from the list including, but not limited to, CD276, CD272, CD152, CD223, CD279, CD274, TIM-3 and B7-H4; or any immune-checkpoint protein antigen antibody taught in the art.

Depending on the nature of the combinatory therapy, administration of the polypeptide therapeutic agents of the invention may be continued while the other therapy is being administered and/or thereafter. The polypeptide therapeutic agents may be administered prior to, concurrently with, or following the additional anti-cancer therapy, usually within at least about 1 week, at least about 5 days, at least about 3 days, at least about 1 day. The polypeptide therapeutic agents may be delivered in a single dose, or may be fractionated into multiple doses, e.g. delivered over a period of time, including daily, bidaily, semi-weekly, weekly, etc. The effective dose will vary with the route of administration, the specific agent, the dose of anti-cancer agent, and the like, and may be determined empirically by one of skill in the art.

In various embodiments, the patient previously responded to treatment with an anti-cancer therapy, but, upon cessation of therapy, suffered relapse (hereinafter “a recurrent proliferative disease”).

In various embodiments, the patient has resistant or refractory cancer. In various embodiments, the cancer is refractory to androgen deprivation therapy. In various embodiments, the cancer is refractory to AR targeted therapy. In various embodiments, the cancer is refractory to hormone deprivation therapy. In various embodiments, the cancer is refractory to immunotherapy treatment. In various embodiments, the cancer is refractory to treatment with a chemotherapeutic agent. In various embodiments, the cancer is refractory to treatment using depleting antibodies to specific tumor antigens. In various embodiments, the cancer is refractory to treatment using agonistic, antagonistic, or blocking antibodies to co-stimulatory or co-inhibitory molecules (immune checkpoints). In various embodiments, the cancer is refractory to targeted treatment with an immunoconjugate, antibody-drug conjugate (ADC), or fusion molecule comprising a depleting antibody to a specific tumor antigen and a cytotoxic agent. In various embodiments, the cancer is refractory to targeted treatment with a small molecule kinase inhibitor. In various embodiments, the cancer is refractory to treatment using DHT blockers. In various embodiments, the cancer is refractory to treatment using radiation. In various embodiments, the cancer is refractory to combination therapy involving, for example, two or more of: immunotherapy treatment, treatment with a chemotherapeutic agent, treatment using depleting antibodies to specific tumor antigens, treatment using agonistic, antagonistic, or blocking antibodies to co-stimulatory or co-inhibitory molecules (immune checkpoints), treatment with a immunoconjugate, ADC, or fusion molecule comprising a depleting antibody to a specific tumor antigen and a cytotoxic agent, targeted treatment with a small molecule kinase inhibitor, treatment using surgery, treatment using stem cell transplantation, treatment using DHT blockers and treatment using radiation.

### Pharmaceutical Compositions

In various embodiments, the polypeptide therapeutic agents of the present invention are often administered as pharmaceutical compositions comprising an active therapeutic agent, i.e., and a variety of other pharmaceutically acceptable components. (See Remington's Pharmaceutical Science, 15.sup.th ed., Mack Publishing Company, Easton, Pa., 1980). The preferred form depends on the intended mode of administration and therapeutic application. The compositions can also include, depending on the formulation desired, pharmaceutically acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In various embodiments, pharmaceutical compositions for the treatment of primary or metastatic cancer can be administered by parenteral, topical, intravenous, intratumoral, oral, subcutaneous, intraarterial, intracranial, intraperitoneal, intranasal or intramuscular means.

For parenteral administration, pharmaceutical compositions of the invention can be administered as injectable dosages of a solution or suspension of the substance in a physiologically acceptable diluent with a pharmaceutical carrier that can be a sterile liquid such as water, oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies and/or polypeptides can be administered in the form of a depot injection or implant preparation which can be formulated in such a manner as to permit a sustained release of the active ingredient. Typically, the pharmaceutical compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The polypeptide agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

In various embodiments, methods of the present invention include administering to a patient in need of treatment a therapeutically effective amount or an effective dose of sEphB4-HSA polypeptide of the present invention. In various embodiments, effective doses of the polypeptides of the present invention, e.g. for the treatment of primary or metastatic cancer, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

In various embodiments, the dosage may range from about 0.0001 to 100 mg/kg, and more usually 0.01 to 5 mg/kg, of the host body weight. For example, dosages can be 1 mg/kg body weight or 10 mg/kg body weight or within the range of 1-10 mg/kg. In various embodiments, the dosage of the polypeptide administered to the patient is selected from the group consisting of about 0.5, of about 1.0, of about 1.5, of about 2.0, of about 2.5, of about 3.0, of about 3.5, of about 4.0, of about 4.5, of about 5.0, of about 6.0, of about 7.0, of about 8.0, of about 9.0, and of about 10.0 mg/kg. In various embodiments, the treatment regime entails administration once per every two weeks or once a month or once every 3 to 6 months. Therapeutic entities of the present invention are usually administered on multiple occasions. Intervals between single dosages can be weekly, bi-weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

Toxicity of the polypeptides described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the polypeptides described herein lies preferably within a range of circulating concentrations that include the effective dose with little or no toxicity. The dosage can vary within this range depending upon the dosage form employed and the route of administration utilized. The exact formulation, route of administration and dosage can be chosen by the subject physician in view of the patient's condition. (See, e.g., Fingl et al., 1975, In: The Pharmacological Basis of Therapeutics, Ch. 1).

The following examples are provided to describe the disclosure in further detail.

### Example 1

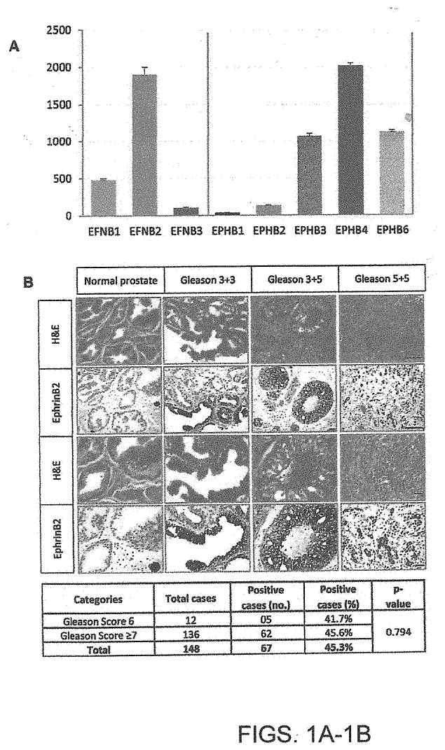

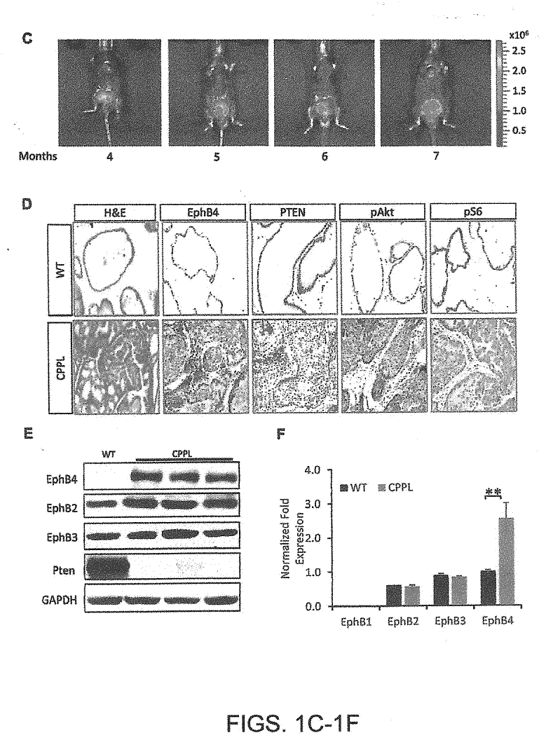

In this example, in order to clarify the role of EphB in PC, EphB-ephrinB gene expression analysis was performed in 492 PC patients in The Cancer Genome Atlas (TCGA) database. It was determined that EphB4 has the highest expression in prostate cancer among EphB receptors, followed by EphB3 and EphB6. EphB2 and EphB1 levels were significantly lower (FIG. 1A). Among the ligands, only ephrinB2, the cognate receptor of EphB4 is expressed at high levels while ephrinB1 and ephrinB3 have very low levels.

EphrinB2 protein expression was then determined in 74 PC samples and 40 normal prostate tissues. EphrinB2 was highly expressed in 58% of the prostate cancer tissues Tumor cells, tumor vessels, and stroma all stained positive for ephrin B2 while all of the 40 normal prostate glands and normal vessels were negative (FIG. 1B). There was no correlation between Gleason grade and ephrinB2 expression. The correlation of Gleason grade and ephrinB2 expression was then analyzed. Surprisingly, ephrinB2 did not increase with increasing Gleason grade and stage, indicating that ephrinB2 is upregulated in all tumor grades and stages of prostate cancer (see Table 1).

TABLE 1
ephrinB2 immunohistochemical expression in
different grade groups of PC

| | Total | Positive | Positive | |
|---|---|---|---|---|
| | Cases | Cases (#) | Cases (%) | p-value |
| Grade Group 1 | 12 | 05 | 41.7% | 0.562 |
| (Gleason Score 3 + 3 = 6) | | | | |
| Grade Group 2 | 28 | 11 | 39.3% | 0.562 |
| (Gleason Score 3 + 4 = 7) | | | | |
| Grade Group 3 | 10 | 06 | 60.0% | 0.562 |
| (Gleason Score 4 + 3 = 7) | | | | |
| Grade Group 4 | 46 | 18 | 39.1% | 0.562 |
| (Gleason Score 8) | | | | |
| Grade Group 5 | 52 | 27 | 51.9% | 0.562 |
| (Gleason Score 9-10) | | | | |
| Total | 148 | 67 | 45.3% | 0.562 |

### Example 2

In this example, experiments were performed to determine whether EphB4 and ephrinB2 expression is regulated by Pten loss of function and thus activation of the PI3K pathway. Specifically, a conditional Pten^{-/-} luciferase (cPten^{-/-}L) reporter under the prostate-specific probasin promoter was performed (see Methods and Materials section below). Luciferase expression allows real time live mouse tumor imaging while probasin driven Cre leads to the Pten allele deletion. PC development begins at 9-weeks of age when testosterone begins to rise. The growth of primary prostate cancer was followed noninvasively by bioluminescence imaging (BLI)(R. S. Liao et al., Transl Androl Urol, 2(3):187-196, 2013). Beginning at 6 to 8 weeks of age, a cohort of 45 mice was monitored using BLI at intervals of 2 weeks for up to 52 weeks. Luciferase expression representing prostate cancer burden increased over time (FIG. 1C). A change in the bioluminescent signal from blue to green to red indicates increasing tumor burden. EphB4 expression was measured in mouse prostate gland by immunohistochemistry (IHC), and western blotting. An increase in EphB4 expression was seen in the tumor, while normal prostate gland had no expression (FIGS. 1D and 1E). The expression of other EphB receptor family members (EphB1, EphB2, EphB3) was also examined. EphB2 did not show an appreciable change by western blot (FIG. 1E), instead quantitative PCR showed downregulation of EphB2 with concurrent increase in EphB4 at the mRNA level in Cre-Pten^{-/-}-Luciferase (CPPL) mice. The EphB1 level was undetectable via quantitative PCR (FIG. 1F). Furthermore, there were no significant change in EphB3 level between tumor and normal tissues (FIGS. 1E and 1F). Thus, Pten loss and induction of PI3K appear to express EphB-ephrinB2 receptor-ligands similar to the human PC.

### Example 3

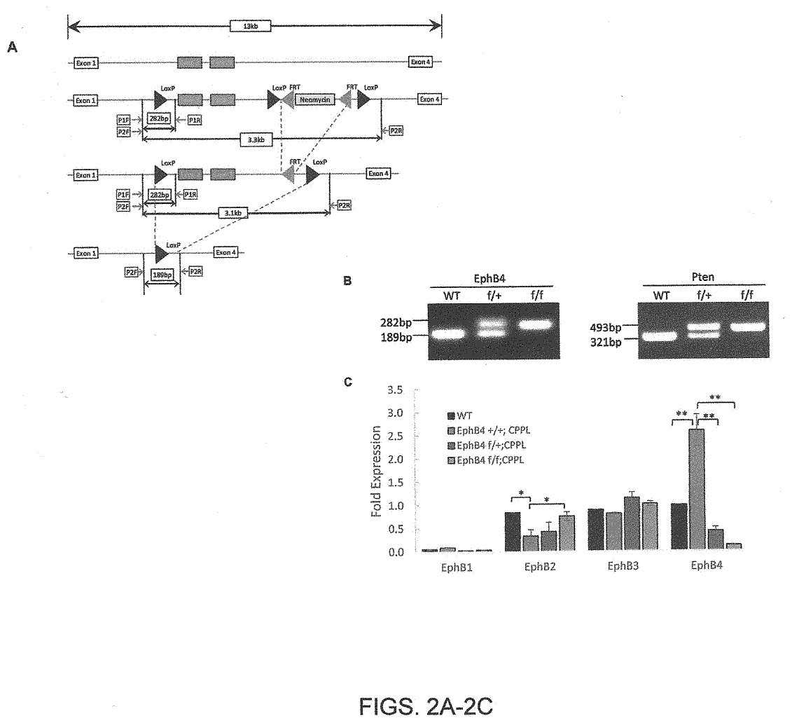

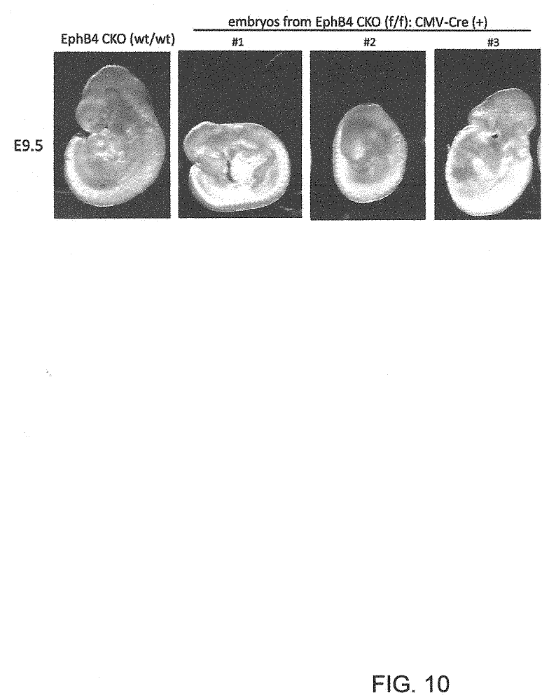

In this example, studies were performed to determine whether EphB4 is required for the initiation and progression of prostate cancer in Pten null mouse prostate cancer. A floxed allele of EphB4 for conditional EphB4 knockout (EphB4 CKO) mice in prostate epithelium similar to the Pten deletion was generated (FIG. 2A). Genotyping confirmed wild type EphB4 (EphB4^{wild-type/wild-type}: 189 bp), heterozygous (EphB4^{fox/wild-type}: 189 bp and 282 bp) and homozygous (EphB4^{flox/flox}: 282 bp) floxed EphB4 (FIG. 2B). To check the phenotype of condition EphB4 deletion, EphB4^{flox/flox} (EphB4^{f/f}) mice were cross-bred with CMV-Cre mice for global gene deletion. Embryonic whole mouse homozygous deletion of EphB4 displayed morphologic defects at E8.5 and embryonic lethality by E9.5-E10.5 (FIG. 10) consistent with embryonic lethality at E9.5-10.5 by classic EphB4 knock out reported previously (Gerety et al., Mol Cell, 4(3):403-414, 1999), while heterozygous embryos had no apparent defects. To study the role of EphB4 in adult life, whole mouse conditional deletion was performed in 12-week-old mice by cross-breeding EphB4^{f/f} with tamoxifen-inducible CMV-Cre mice. Fifteen mice were monitored for 20 months. There was no apparent phenotype. Multiple organ analysis including heart, lung, liver, kidney, colon, brain collected at necropsy showed no abnormalities on histologic examination (data not shown). Taken together these data suggest that EphB4 is critically required in embryonic development but not in adult life.

Next, the expression of EphBs in Ephb4^{f/f}; CPPL prostate tissues was determined. EphB4 RNA level was significantly reduced in EphB4 heterozygotes with even greater decline in homozygotes knockout CPPL mice (FIG. 2F).

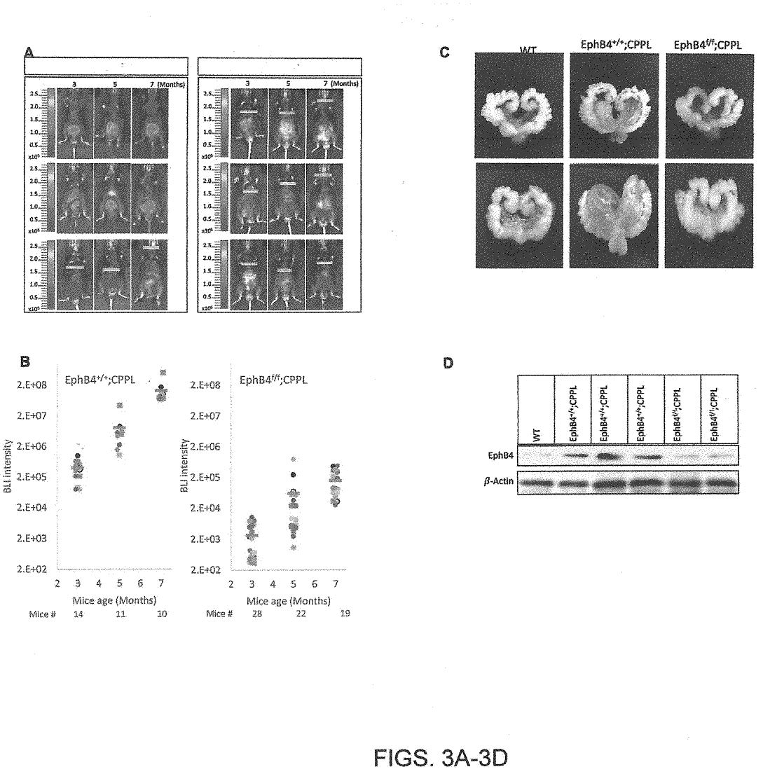

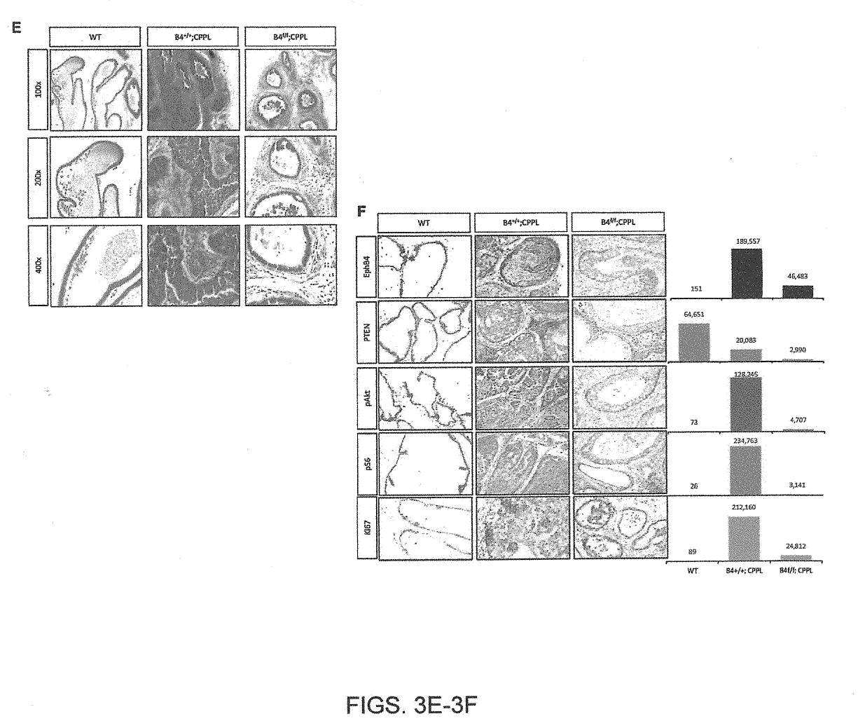

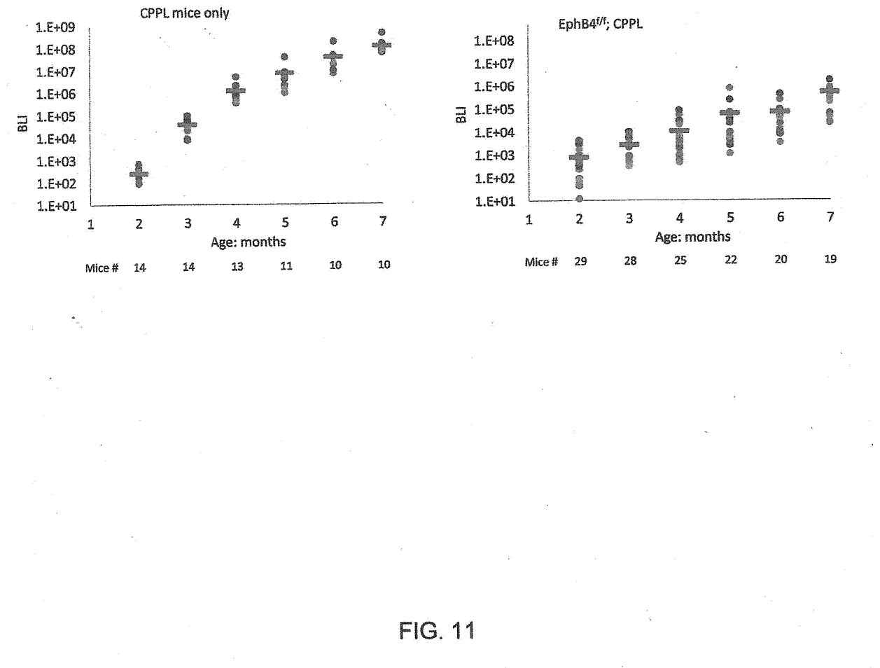

In order to study the role of EphB4 in prostate cancer development in the context of Pten deletion, Ephb4 floxed mice were crossed with CPPL mice. Mice were monitored over 7 months. Ephb4 deletion prevented the development of PC in 16 of 29 mice, and minimal tumor development was observed in the other 13 mice (FIGS. 3A, 11). Ephb4 knock out mice had no or very slow tumor progression as shown by the markedly low levels of luciferase in these 29 mice, compared to marked increase in the signal intensity in 14 control (Pten^{-/-};Ephb4^{-/-} vs Pten^{-/-};Ephb4^{+/+}) mice (FIG. 3B) assessed by quantitative measurement of luciferase signal. These observations were consistent with the results of prostate tissue analysis. Prostate glands developed large bilateral tumors in EphB4 wild type Pten null mice (Ephb4^{+/+}; CPPL) and Ephb4 heterozygote Pten null animals (Ephb4^{+/+}; CPPL). In contrast, Ephb4 and Pten knockout genotypes (Ephb4^{f/f}; CPPL) had normal appearing prostates (FIG. 3C). Tumors harvested from the prostate gland of Pten null mice (Ephb4^{+/+}; CPPL) showed elevated levels of EphB4, but only a minimal signal was obtained in wild type (Pten^{+/+}; Ephb4^{+/+}) and Ephb4 knock out CPPL mice (Ephb4^{f/f}; CPPL) prostate gland (FIG. 3D). Pathologic analysis of the prostate gland showed near normal appearing glandular structures, but some increase in cellularity in the glandular structures in Ephb4 deficient mice, compared to dense tumor with loss of normal architecture in the Pten knock out mice (FIG. 3E). Increased apoptosis and low proliferation in the glandular structures was evident in the EphB4 null group (CPPL; Ephb4^{f/f};) compared to the Ephb4 wild type group (FIG. 3F). Tissue analysis of Ephb4 knock out Pten null mice showed marked reduction in PTEN, EphB4, phosphorylated AKT (pAKT), and phosphorylated S6 (pS6) (FIG. 3G). PTEN was expressed in normal prostate tissue but absent in CPPL mice. Moreover, the Ephb4 null group showed low proliferation by Ki67 immunohistochemistry in the glandular structures compared to Ephb4 wild type group (FIG. 3F).

### Example 4

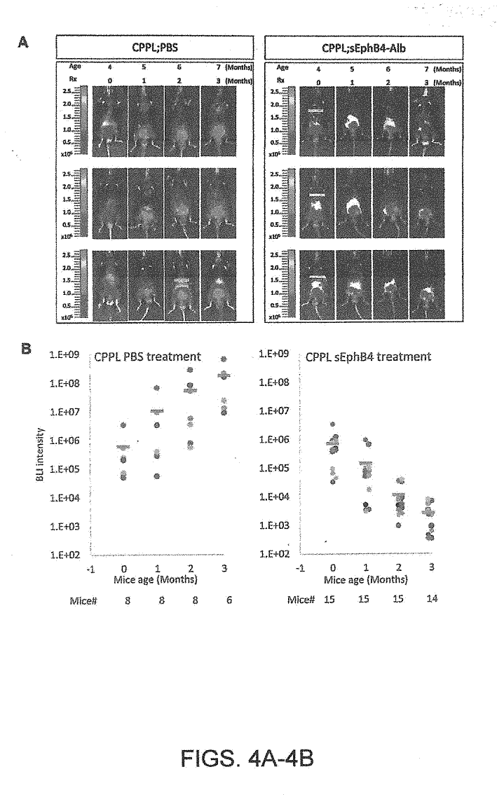

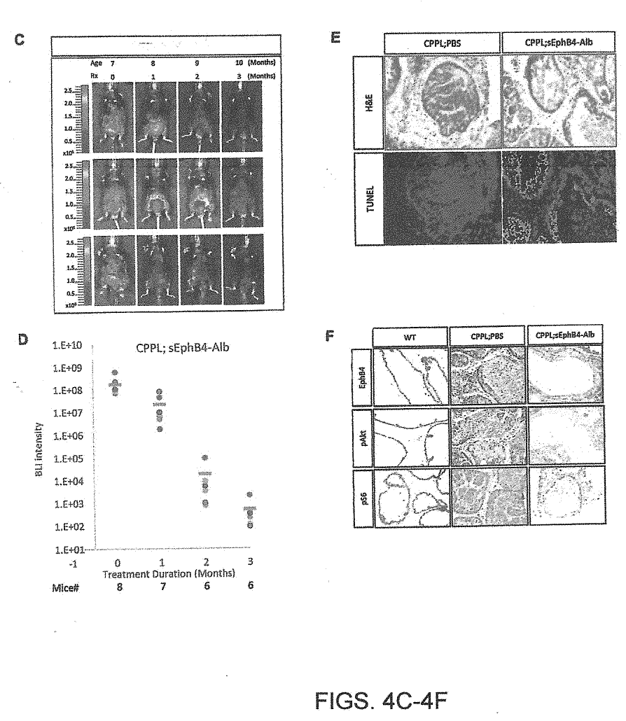

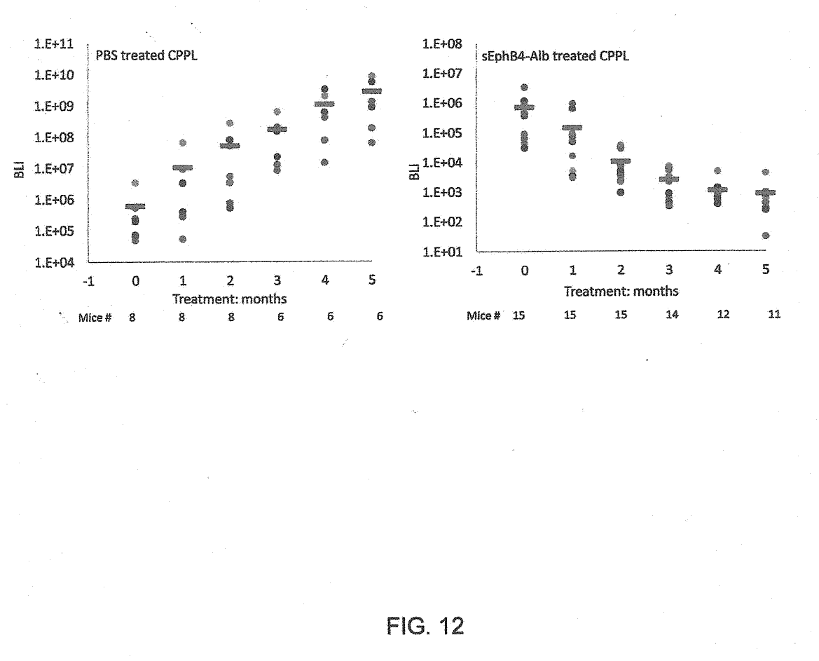

In this example, studies were performed using a decoy soluble EphB4 receptor to block EphB4-ephrinB2 bidirectional signaling. Soluble EphB4 (sEphB4) is the full-length extracellular domain of EphB4. sEphB4 is a monomeric protein which binds cognate ligand ephrinB2 with high affinity, and even dissociates the ephrinB2-EphB4 complex. As a result of this binding, sEphB4 blocks interaction of endogenous ephrinB2 to endogenous EphB4, and other EphB receptors, thus inhibiting bidirectional signaling. sEphB4 has activity in many PC xenograft model; however, sEphB4 activity in a genetic mouse model has not been tested. A murine version of the protein consisting of full-length extracellular domain of EphB4 and full-length murine albumin was engineered in frame at the C-terminus thus producing a fusion protein (sEphB4-Alb) of 129 kD, which has a half-life of over 24 hr in mice. The activity of sEphB4-Alb in preventing tumor development in CPPL mice was investigated. sEphB4-Alb therapy was started at age 8 weeks, prior to tumor initiation which begins at adolescence (week 12 onwards) and continued until age 7 months. Treatment was administered intraperitoneally (IP) three times a week. Tumor development was markedly reduced in all 15 treated mice when measured by quantitative BLI. On average, BLI in the treated group was near 70,000-fold lower than the control group at 3 months of therapy (P<0.05) (FIGS. 4A and 4B, 12).

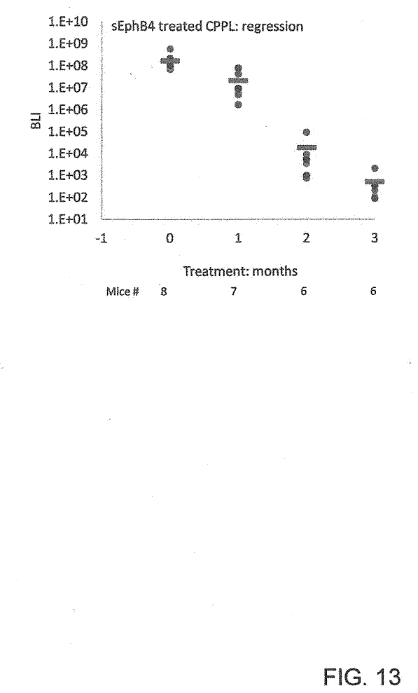

Having demonstrated the effectiveness of sEphB4-Alb in the Pten null model, an efficacy study to determine if sEphB4-Alb can induce regression of established tumors or retard further progression was performed. 16 mice with established prostate tumors documented by luciferase imaging with either sEphB4-Alb (n=8) or PBS controls (n=8) were treated. Mice were treated for 3 months and BLI was performed every 4 weeks. Tumor regression in all 8 mice in the drug treatment group was observed, with complete regression in 5 and near complete regression in the remaining 3 (FIGS. 4C and 4D, 13). As a result of treatment, the average BLI signal went down from 1.68E+08 to 5.13E+02 (>300,000 times in the treatment cohort). Prostate glands were harvested at the end of the study and analyzed for signaling pathway components downstream of PI3K. Increased apoptosis was observed in the EphB4 null group in the glandular structures compared to the EphB4 wild type group (FIG. 4E). Both pAKT and pS6 were markedly lower in the treated compared to control group (FIG. 4F).

### Example 5

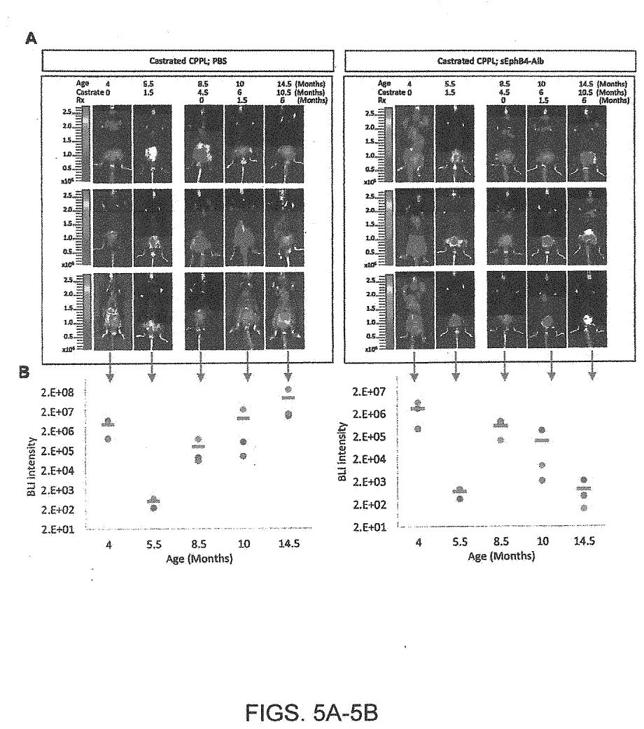

The biggest unmet need in PC is therapy for advanced tumor refractory to androgen deprivation. To assess the efficacy of sEphB4-Alb in a comparable population, androgen-independent tumors in Pten null mice by castration after the tumors were established at 12 weeks of age were generated. Mice were monitored every four weeks for tumor regression with castration followed by recurrence. Initially, the bioluminescent signal declined in the first 4-6 weeks of castration with eventual recurrence of androgen-independent tumor over a period of 10-12 weeks. Mice (3 per group) were then treated either with PBS or sEphB4-Alb. All sEphB4 treated mice had decline in BLI signal while signal continued to increase in control group with an average fold change of over 10 thousand times (or 10⁴) in controls compared to the drug therapy group (average 8.96E+02 in sEphB4-Alb group versus average 9.11 E+07 in control group after 6-month treatment) (FIGS. 5A and 5B). Taken together, the data suggest that sEphB4-Alb is effective in androgen-independent tumors. The present inventor was somewhat surprised to see the efficacy in established tumors in the pre-castration group, and even more so in the castration group. While sEphB4-Alb inhibits PI3K pathway, the present inventor would have expected tumor escape through the AR pathway. Based on this data, it was decided to explore the mechanism of action by studying EphB4 knock down in PC cell lines in vitro including hormone independent variants.

### Example 6

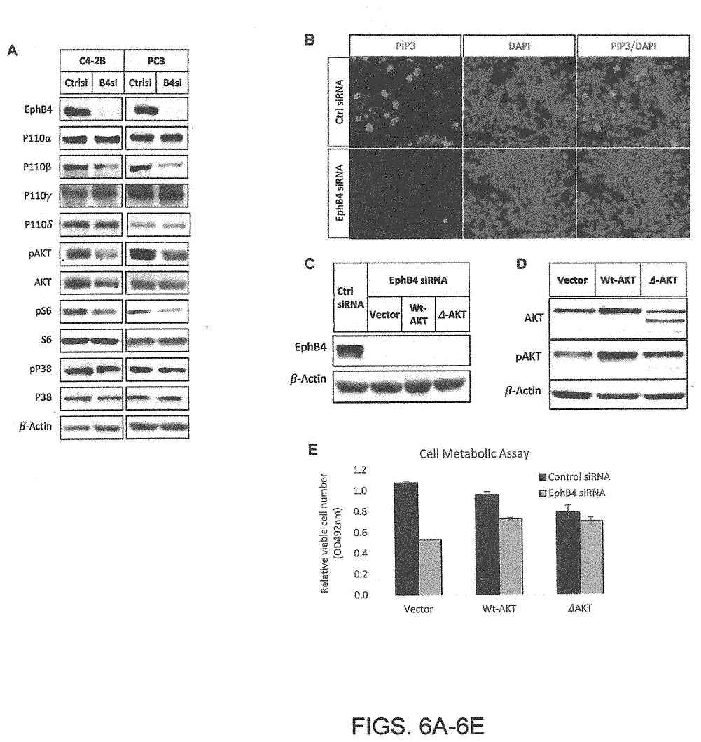

Activation of the PI3K-AKT pathway through growth factor receptors including epidermal growth factor (EGF)-EGFR upregulates EphB4 expression (Kumar et al., Cancer Res, 69(9):3736-3745, 2006). Furthermore, activation of EphB4 receptor with clustered ephrinB2-Fc increased phospho-AKT levels indicating a positive feedback loop. This is now validated in the genetic mouse model of Pten knock out prostate cancer. Prostate tumor in this model showed induction of EphB4 and knock out EphB4 markedly reduces the risk of PC with attenuation of PI3K pathway activation markers. The present inventor wished to determine if knock down of EphB4 lowers the levels of PI3Ks in prostate cancer cell lines such as PC3 and castrate-resistant prostate cancer cell lines (C4-2B and 22Rv1). Silencing EphB4 expression via an Ephb4 siRNA (Xia et al., Oncogene, 25(5):769-780, 2006) lowered EphB4 and the PI3K downstream markers, phosphorylated AKT (pAKT, Thr308) and phosphorylated ribosomal protein S6 (pS6, Ser235/Ser236; FIG. 6A). Notably, EphB4 siRNA specifically down-regulated PI3K p110 subunit β, but not p110 α, γ and δ in both PC3 and C4-2B cell lines. PI3K p110 β and δ isoforms promote PC development and metastasis in several in vitro models and their expression in clinical prostate cancer specimens is associated with relapse after surgery. The biological function of PI3K to convert phosphatidylinositol (3,4)-diphosphate (PIP2) to phosphatidylinositol (3,4,5) triphosphate (PIP3) was also diminished with EphB4 knock down (FIG. 6B) indicating that EphB4 plays an important role in PI3K activity. EphB4 knock down however had no effect on total or activated form of MAPK and p38 (FIG. 6A). Thus, EphB4 function is specific to the PI3K pathway. EphB4 knock down was shown to inhibit PC cell growth in vitro (Kertesz et al., 2006; Xia et al., 2006). To test the specificity of EphB4 siRNA in inhibiting PI3K activity, the cell viability rescue assay with ectopic expression of wild-type AKT and the constitutionally active form of AKT (A-AKT) (Kohn et al., J Biol Chem, 271(36): 21920-21926, 1996) (FIGS. 6C, 6D) was performed. Both wild-type AKT and A-AKT rescued PI3K inhibition from EphB4 siRNA (FIG. 6E). These data further confirmed that EphB4 regulates PI3K pathway at or above AKT level.

In order to determine whether EphB4 regulates the pathway at the PI3K level, the expression levels of PI3K catalytic p110 isoforms. EphB4 siRNA specifically down-regulated PI3K p110 subunit β, but not p110 α, γ and δ in both PC3 and C4-2B cell lines was examined. PI3K p110 (3 and S isoform promote PC development and metastasis and are associated with biochemical relapse after surgery when expression in prostate tumors (Hill et al., Prostate, 70(7), 755-764, 2010). Taken together, the data indicate that EphB4 regulates PI3K pathway at the PI3K p110p level in the cell lines examined.

### Example 7

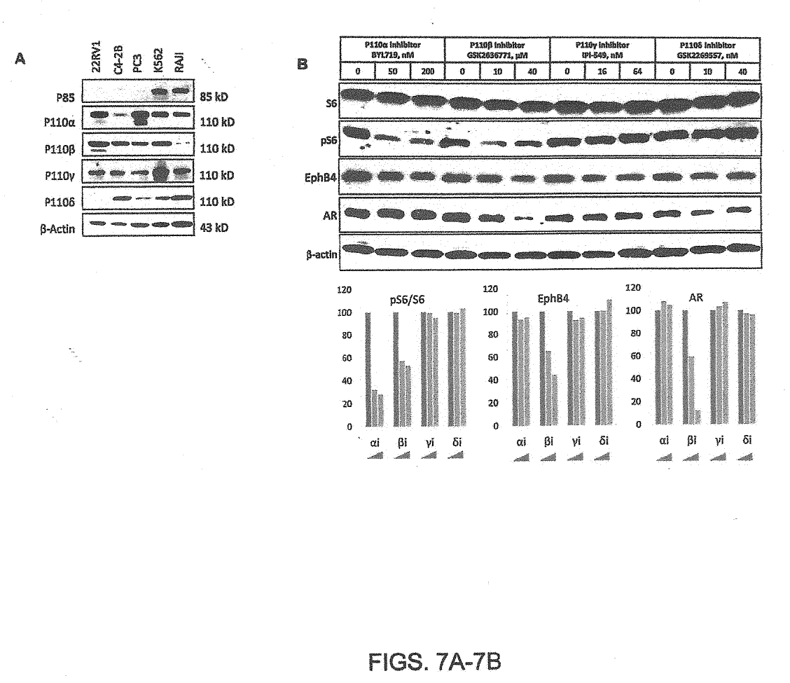

EphB4 knockdown reduces levels of PI3K isoform p110β in vitro in PC cell lines. It was next determined whether EphB4 regulates other PI3K catalytic isoforms. Since 22RV1 and C4-2B express PI3K p110α and β but low levels of other isoforms, hematopoietic leukemia and lymphoma cell lines K562 (Erythroleukemia cell line) and Raji (Burkitt's lymphoma cell line) that express high levels of p110γ and p110δ were included as positive controls (FIG. 7A). Specific inhibitors of each PI3K isoform for inhibition of the downstream signal, phosphorylated S6 were then tested. Inhibitors of PI3K p110 α (BYL719) and β isoforms (GSK2636771) significantly reduced pS6 levels but not total amount of S6. No change was observed in pS6 levels with inhibitors of p1107 (IPI-549) or p1108 (GSK2269557) (FIG. 7B).

It has previously been suggested that p110p induces androgen receptor (AR) and AR downstream signaling and that specific inhibition of p110β reduces AR levels (Hill et al., Prostate, 70(7), 755-764, 2010). The present inventor observed that inhibition of p110β reduced EphB4 and AR levels while inhibitors of p110α/7/S had no effect on either protein (FIG. 7B). It is thus possible that EphB4 induction through PI3K signals through p110β in PC leads to tumor initiation and progression. Targeting EphB4 inhibits p110β to induce loss of cell viability with potential escape through AR induction.

### Example 8

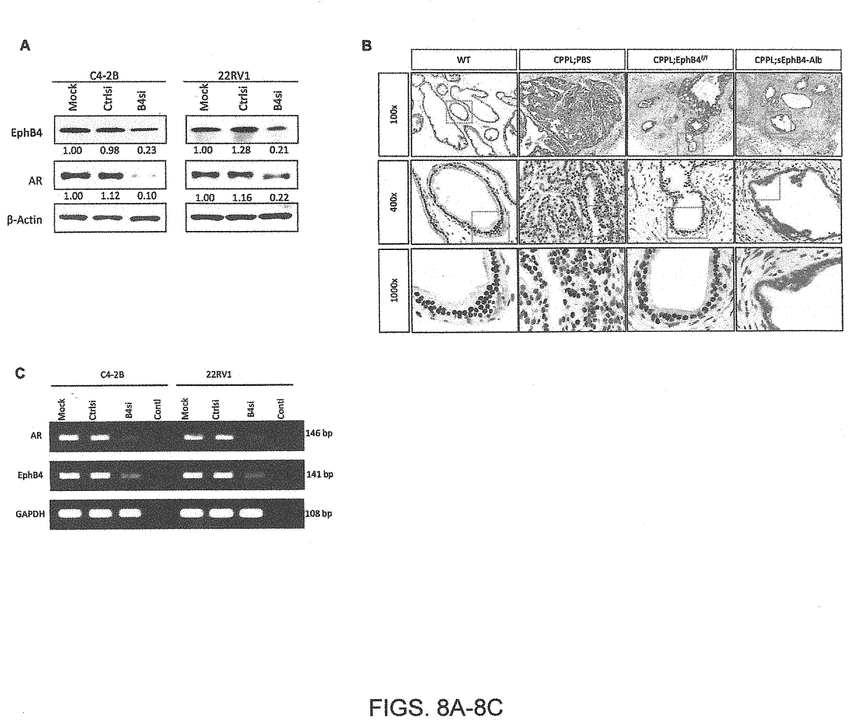

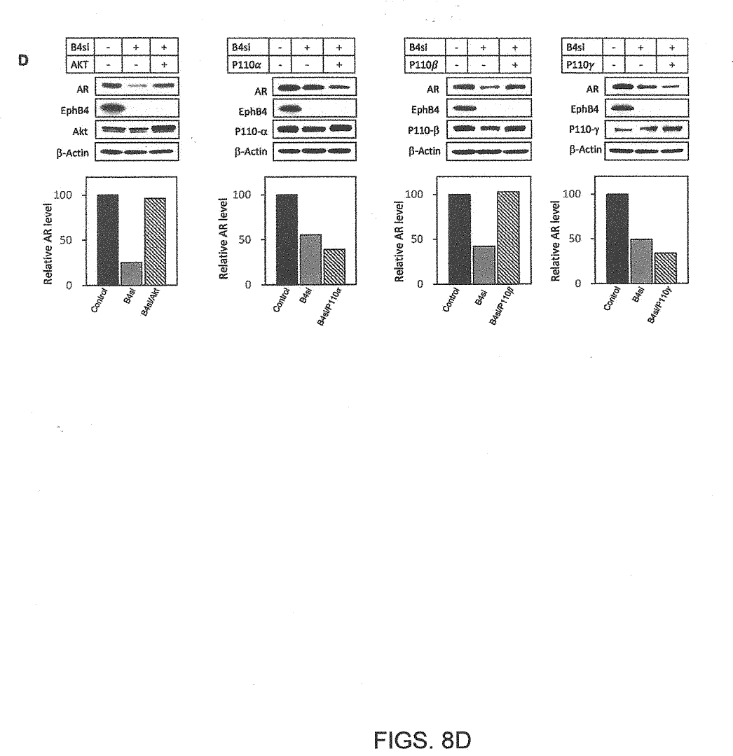

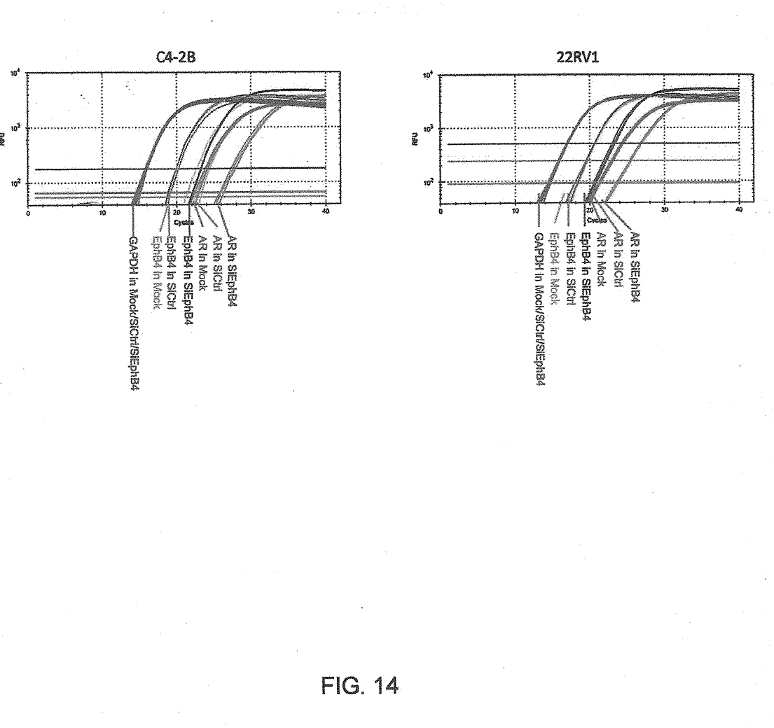

To determine whether EphB4-loss reduces AR levels, experiments to check AR protein levels with EphB4 knockdown were performed. EphB4 knockdown with siRNA markedly reduced AR levels in castration-resistant PC cell lines (FIG. 8A) supporting the role of EphB4 in AR expression. Further, in vivo studies using the EphB4-ephrinB2 competitive inhibitor sEphB4-Alb in CPPL mice were performed. Androgen receptor mRNA levels were reduced by 80% in drug treated mice compared to control mice (FIG. 8B) and 84% in EphB4 knockdown cell line via quantitative PCR (FIGS. 8C, 14). Based on the above findings that PI3K isoforms have distinct functions on AR protein and mRNA levels, the present inventor sought to determine whether EphB4 regulates AR through PI3K pathway. Rescue experiments with AKT and each PI3K isoform in the context of EphB4 knockdown were conducted. Interestingly, AR expression was rescued with ectopic expression of AKT and PI3K p110β subunits but not a and γ (FIG. 8D), indicating that EphB4 regulates AR through PI3K pathway in particular p110.

### Example 9

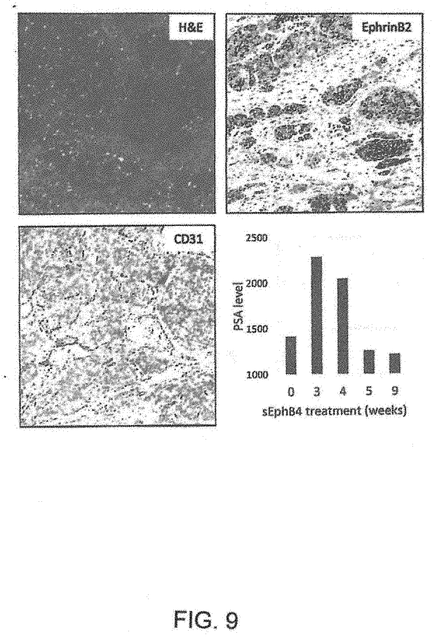

sEphB4-HSA has been tested in a human phase I trial with acceptable safety even over prolonged therapy (El-Khoueiry et al., European Journal of Cancer, 69, S11, 2016; Hasina et al., Cancer Res, 73(1):184-194, 2013; Thomas et al., Journal of Clinical Oncology, 36(4):285-285, 2018). While preparing to conduct clinical trials in prostate cancer, the present inventor had the opportunity to offer compassionate treatment to a subject with castration-resistant prostate cancer (CRPC). A 68-year-old man with CRPC with extensive bone, bone marrow, and visceral metastases had failed 9 prior regimens including ADT, AR pathway inhibition (enzalutamide and abiraterone), radiation, chemotherapy (docetaxel, cabazitaxel and carboplatin), sipuleucel-T and radium-223. The tumor was analyzed and found to have high EphB4 and ephrinB2 expression (FIG. 9). Single patient IND approval was obtained on a compassionate basis. After IRB and FA approval, he was treated with sEphB4-Alb at a dose of 10 mg/kg weekly intravenous infusion for 3 weeks. PSA a surrogate of activity was 1416 at initiation of therapy increased continued to increase in the first 3 weeks to 2439, followed by steady decline to 1200 and remained low for the ensuing 6 weeks, indicating there was biological activity of sEphB4-HSA.

Based on the data and observations described herein, it appears EphB4 and ephrinB2 are highly expressed in PCs and the experiments described herein suggest a significant role for the EphB4-ephrinB2 pair through regulation of PI3K and AR signaling. Given the central role of AR and PI3K in PC, sEphB4 offers a novel approach to targeting PCs, most specifically, PCs which are PTEN deficient and/or PCs which are refractory to AP targeted treatment.

The human form of sEphB4-HSA has now been advanced to the clinic and can be administered for prolonged periods of time as a single agent and in combination with other agents with acceptable toxicity. The present inventor will be soon launching clinical trials in advanced disease PC with the belief that sEphB4-HSA may provide potential benefit in this highly unmet medical need.

### Methods and Materials

### PTEN-Null Prostate Cancer Mouse Model

The prostate specific PTEN knockout (Cre-PTEN^{-/-}-Luc, CPPL) mouse model was kindly provided by Dr. Pradip Roy-Burman and described previously (C. P. Liao et al., Cancer Res, 67(15):7525-7533, 2007). Mouse models of prostate adenocarcinoma with the capacity to monitor spontaneous carcinogenesis by bioluminescence or fluorescence. Cancer Res, 67:7525-33, 2007). Cre-PTEN−/−Luc mice were randomized into two groups (n=4) and administered intraperitoneally with either 20 mg/kg soluble EphB4-Albumin or Phosphate-buffered saline (PBS), twice a week and prostate tumors were monitored by biluminescence imaging (Xenogen) before treatment and every 4 weeks after treatment.

### Biluminescence Imaging (BLI)

Mice were given a single i.p. injection of ketamine (50 mg/kg) and xylazine (10 mg/kg) followed by i.v. injection of luciferin (50 mg/kg). After waiting for 4.5 min to allow proper distribution of luciferin, the mice were placed in the chamber of an IVIS 200 optical imaging system (Xenogen Corp.). Photons were collected for a period of 1 min, and images were analyzed using LIVING IMAGE software v. 2.50 (Xenogen). Signal intensity was quantified for defined regions of interest as photon count rate per unit body area per unit solid angle subtended by the detector (units of photons/s/cm2/steradian).

### Generation and Genotyping of the Conditional EphB4 Knockout Mice

Based on the known gene structure of EphB4, a gene targeting vector was constructed to replace exons 2 to 3 and parts of intron 1-4 by a pEZ FRT Lox cassette (a gift from Dr. Robert Maxson lab) in ES cells. First Loxp inserted into intron 1-2, second Loxp inserted into intron 3-4, the deletion is 33167 bps, from HindIII to AccI including part of intron1-2 (2188 bps), exon 2 (71 bps), intron 2-3 (120 bps), extron3 (288 bps), part of intron 3-4 (598 bps). The lacZ gene was fused in-frame with the EphB4 coding sequence at the start of exon 2. Two correctly targeted ES cell lines were identified by PCR and Southern blot analysis and used to generate chimeric mice. After germline transmission, heterozygote mice are crossed with FLP transgenic mice to remove Neo. Then, Neoless heterozygote mice are crossed with wild type C57BL/6 background mice to remove FLP.

Mice homozygous for floxed EphB4 exon 2-3, EphB4^{floxP/floxP}, were crossed with the CMV-Cre strain were obtained from the Jackson Laboratory (Bar Harbor, Me.) and PB-Cre-Pten-Luc mice, in which the Cre transgene is controlled by a modified probasin promoter (ARR2PB). Generation of a prostate epithelial cell-specific Cre transgenic mouse model for tissue-specific gene ablation. Mech Dev 101: 61-69]. Littermate controls lacking the Cre transgene were used in all experiments. All procedures were approved by Institutional Animal Care and Use Committee and performed in accordance with the Animal Welfare Act regulations

Mice were genotyped by PCR as described previously. Mouse tail-tips were isolated and incubated overnight at 55° C. in lysis buffer (Cat #102-T, VIAGEN Biotech, LA, CA) with 0.5 μg/mL proteinase K (Cat #03-H5-801-001, Roche Diagnostics, Indianapolis, Ind.). Tail-tip samples were then incubated at 85° C. for 45 min before use. The forward primer1 (5′-TTCTCGCCTGCGCTACCTGAATG-3′) and the reverse primer2 (5′-ACCAGGGCTCCATTTCTAGGTCG-3′) were used to distinguish the wild type and target alleles by amplifying the flanking loxP sites. The forward primer (5′-GATCCTGGCAATTTCGGCTAT-3′) and the reverse primer (5′-TTGCCTGCATTACCGGTCGAT-3′) were used to detect the Cre transgene. Genomic DNA fragments were amplified at 95° C. for 5 min, then 95° C. for 45 sec, 58° C. for 40 sec, and 72° C. for 60 sec for 36 cycles, then 72° C. for 5 min. For detection of exon 5 deletion, genomic DNA samples were isolated from different mouse organs using similar methods as for mouse tail-tips. The forward primer (5′-TAGGCTGGGCAGTGCTGTTCTGG-3′), and reverse primer, (5-CTCCTGTAGTCCAAGCTGGTCTC-3′) were used to detect exon 2-3 deletion.

### Antibodies and Other Reagents

Antibodies against P110α, β110β, P110γ, P110δ, p85 (PI3K subunits, rabbit monoclonal), Akt, phosphorylated Akt (Thr308; Ser473), S6, phosphorylated S6 (Ser240/244), ERK1/2 (Thr202/Tyr2O4), phosphorylated p38, p38, androgen receptor, PTEN were from Cell Signaling (Danvers, Mass.). β-actin was from Sigma (St Louis, Mo.) and GAPDH (mouse monoclonal) antibody was from Millipore (Temecula, Calif.). Ki67 antibody was from Abcam (Cambridge, Mass.) and anti-Ptdlns(3,4,5)P3 was from Echelon Biosciences (Salt Lake City, Utah). Antibodies to Eph receptors and ligands were obtained from R&D Systems (Minneapolis, Minn.). EphB4 antibody was from VasGene Therapeutics (Los Angeles, Calif.). Horse radish peroxidase (HRP) and IRDye conjugated secondary antibodies were from Rockland (Gilbertsville, Pa.).

Complementary (cDNA) encoding of mouse EphB4 representing the entire extracellular domain was cloned upstream of the mature mouse serum albumin pCRscript and placed into the mammalian expression vector under control of the cytomegalovirus (CMV) promoter stably expressed in the Chinese hamster ovary (CHO) cell line. The expressed sEphB4-Alb fusion protein was purified to homogeneity as described previously (Scehnet, Blood 2009 113:254-263).

Prostate adenocarcinoma tissue microarray, containing 80 cases of adenocarcinoma, 8 adjacent normal prostate tissue and 8 normal prostate tissue, duplicate cores per case obtained from Biomax #PR1921b (Derwood, Md.).

### Western Blotting

For Western blot, typically 20 μg of whole-cell lysates were run on 4-20% Tris-glycine gradient gel (Bio-Rad, Hercules, Calif.) and transferred onto nitrocellulose membrane (Bio-Rad, Hercules, Calif.). The membrane was blocked with 5% non-fat dry milk in TBS and 0.05% Tween-20 (TBST) for 40 min, and then incubated with 1 μg/ml primary antibody at 4° C. overnight. Membrane was washed three times for 10 min each and incubated with secondary HRP-labeled or IRDye labeled secondary antibody for 40 min. After three times wash with TBST, HRP signal was detected using Femto Maximum Sensitivity chemiluminescent substrate from Thermo Scientific, and IRDye signal was detected by Odyssey (LICOR, Lincoln, Nebr.).

### Immunofluorescence and Immunohistochemistry

For immunofluorescence, fresh frozen tissue embedded in OCT was sectioned at 5 μm and fixed in phosphate-buffered 4% paraformaldehyde and washed in PBS. Sections were then blocked with goat serum and incubated with primary antibody overnight at 4° C. After washing in PBS, antibody binding was localized with AlexaFluor conjugated appropriate secondary antibodies (Invitrogen, Carlsbad, Calif.). Nuclei were counterstained with DAPI. Images were obtained with a Nikon Eclipse 80i fluorescence microscope and Meta Morph imaging series system. Tissues were also processed for apoptosis analysis with TdT-mediated dUTP nick-end labeling (TUNEL) assay kit (Promega, Madison, Wis.) following manufacturers' instructions.

For immunohistochemistry, the frozen sections were fixed with 3% formaldehyde for 15 minutes at room temperature, following by two PBS washes. The sections were treated with 3% H₂O₂ for 10 min, blocked with goat serum for 1 hour, and incubated with primary antibody for overnight at 4° C. The sections were then washed with PBS and processed with ABC kit (Vector labs, Burlingame, Calif.). The images were obtained with an Olympus BX51 microscope and Image-pro plus 6.0 system.

Four representative pictures were taken for each sample and quantification was performed with Image J (NIH). P value was determined by an unpaired 2-tail student T-test.

### Cell Lines and Culture

PC3 cell lines were obtained from the American Type Culture Collection. C4-2B cell was kindly provided by Michael Stallcup (University of South California), and 22Rv1 and K562 cell lines were kindly provided by Dr. Akil Merchant (University of South California). All these cells were propagated in RPMI-1640 supplemented with 10% FBS, 100 units/mL of penicillin, and 100 μg/mL streptomycin from Cellgro. These cell lines have been validated by HLA typing and molecular phenotyping relative to the respective primary tumors.

### In Situ Hybridization

In situ hybridization was performed as described previously with modifications (Drummond, I. A. et al. Early development of the zebrafish pronephros and analysis of mutations affecting pronephric function. Development 125, 4655-4667 (1998). Specifically, section in situ was performed using ISH kit (Biochain, Hayward, Calif.) according to the manufacturer's protocol. DIG-labeled antisense and sense probes were synthesized by in vitro transcription using T7 and T3 RNA polymerase (Promega, San Luis Obispo, Calif.). A 0.7 kb PCR fragment was amplified from the plasmid (BC076426) containing the full-length cDNA of mouse EphB4 and subcloned into pCR4.0 TOPO vector (Invitrogen, Carlsbad, Calif.). Images were obtained using an Olympus BX51 microscope equipped with a QImaging Retiga 2000R camera. Quantitative RT-PCR

Total RNA was extracted using RNeasy mini kit (Qiagen, Valencia, Calif.) from mouse prostate tissue. First-strand cDNA was synthesized from 2 μg of total RNA with the kit from Fermentas and then quantitative PCR was performed on the MX3000P real-time PCR system (Stratagene, La Jolla, Calif.) using Brilliant II SYBR Green QPCR Mastermix (Stratagene, La Jolla, Calif.) according to the manufacturer's instructions. All reactions were performed in triplicate. The amplification signals were normalized to β-actin. For assessing EphB4 siRNA knockdown effect, total mRNA was extracted from cultured cells transfected with EphB4 siRNA or 3-base mis-match control siRNA (Control siRNA). Primer sequences of studied genes are shown in Table 2.

TABLE 2
Real-time RT-PCR primer sequences

| | Forward | Reverse |
|---|---|---|
| Gene | Primer (5′-3′) | Primer (5′-3′) |
| mEphB4 | GCATTCAGCCAAAGTGAGG | GCCGTTTCCAGTTTTGTGTT |
| mEphB2 | GGATGTGCCCATCAAACTCT | CCTTGAAGGTTCCTGATGGA |
| mEphB3 | AGCTCTACTGCAATGGCGAC | TGCTTTGCTTTGTAACTCC |
| | | CA |
| mEphrinB2 | CTGTTGGGGACTTTTGATGG | TTGTCCGGGTAGAAATTTGG |
| mActb | GGCTGTATTCCCCTCCATCG | CCAGTTGGTAACAATGCCAT |
| | | GT |
| hEphB4 | CAGTTCGAGCACCCCAATAT | ACGAGCTGGATGACTGTGAA |
| hAR | GCTAGAAGGCGAGAGCCTA | TTGTAGTAGTCGCGACTCTG |
| hGAPDH | AAGGTGAAGGTCGGAGTCAA | AATGAAGGGGTCATTGATGG |

siRNA and Transfection

EphB4 siRNA (sequence was 5′-CCGGGAAGGUGAAUGUCAA-3′) was synthesized from Qiagen (Valencia, Calif.). Lipofectamine 2000 (Invitrogen, Carlsbad, Calif.) was used for siRNA transfection following manufacturer's instruction.

### AKT Constructs

The constructs wild-type Akt and myrAkt A4-129 (constitutional activated AKT), which contains a src myristoylation signal sequence were as described previously (Kohn A., Takeuchi F., Roth R. A. (1996) J. Biol. Chem. 271, 21920-21926). These constructs were cloned into the pCMV-SPORT6 vector.

### In Vitro AKT and Phosphorylated AKT Rescue Experiment

C4-2B cells were seeded in 24-well plates at a density of 2×10⁴ cells/well in a total volume of 500 μL and 24 hrs later, cells were transfected with EphB4 siRNA or control siRNA. Another 12 hrs later, these cells were further transfected with either AKT/pCMV-SPORT6 full length (BC020530.1, Open Biosystems), constitutional activated AKT/pCMV-SPORT6 or pCMV-SPORT6 plasmid. 2 days after treatment, cell viability was assessed using 3-(4,5-dimethylthiazol-2-yl)-2,5-diphenyltetrazolium bromide (MTT) as described previously (Kumar S R, Singh J, Xia G, Krasnoperov V, Hassanieh L, Ley E J, et al. Receptor tyrosine kinase EphB4 is a survival factor in breast cancer. Am J Pathol 2006; 169(1):279-93). Protein expression was confirmed by immunoblotting.

### Statistical Analysis

The statistical significance of differences in different samples or groups was determined using an unpaired two-tailed Student t test. Results were considered significantly different if the P value was less than 0.05.

All of the articles and methods disclosed and claimed herein can be made and executed without undue experimentation in light of the present disclosure. While the articles and methods of this invention have been described in terms of preferred embodiments, it will be apparent to those of skill in the art that variations may be applied to the articles and methods without departing from the spirit and scope of the invention. All such variations and equivalents apparent to those skilled in the art, whether now existing or later developed, are deemed to be within the spirit and scope of the invention as defined by the appended claims. All patents, patent applications, and publications mentioned in the specification are indicative of the levels of those of ordinary skill in the art to which the invention pertains. All patents, patent applications, and publications are herein incorporated by reference in their entirety for all purposes and to the same extent as if each subject publication was specifically and subjectly indicated to be incorporated by reference in its entirety for any and all purposes. The invention illustratively described herein suitably may be practiced in the absence of any element(s) not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### Sequence Listings

The amino acid sequences listed in the accompanying sequence listing are shown using standard three letter code for amino acids, as defined in 37 C.F.R. 1.822.

SEQ ID NO: 1 is the amino acid sequence of human
ephrin type-B receptor precursor (NP_004435.3).
Amino acid residues 1-15 encode a signal
sequence.
(SEQ ID NO: 1)
MELRVLLCWASLAAALEETLLNTKLETADLKWVTFPQVDGQWEELSGLDEE
QHSVRTYEVCDVQRAPGQAHWLRTGWVPRRGAVHVYATLRFTMLECLSLPR
AGRSCKETFTVFYYESDADTATALTPAWMENPYIKVDTVAAEHLTRKRPGA
EATGKVNVKTLRLGPLSKAGFYLAFQDQGACMALLSLHLFYKKCAQLTVNL
TRFPETVPRELVVPVAGSCVVDAVPAPGPSPSLYCREDGQWAEQPVTGCSC
APGFEAAEGNTKCRACAQGTFKPLSGEGSCQPCPANSHSNTIGSAVCQCRV
GYFRARTDPRGAPCTTPPSAPRSVVSRLNGSSLHLEWSAPLESGGREDLTY
ALRCRECRPGGSCAPCGGDLTFDPGPRDLVEPWVVVRGLRPDFTYTFEVTA
LNGVSSLATGPVPFEPVNVTTDREVPPAVSDIRVTRSSPSSLSLAWAVPRA
PSGAVLDYEVKYHEKGAEGPSSVRFLKTSENRAELRGLKRGASYLVQVRAR
SEAGYGPFGQEHHSQTQLDESEGWREQLALIAGTAVVGVVLVLVVIVVAVL
CLRKQSNGREAEYSDKHGQYLIGHGTKVYIDPFTYEDPNEAVREFAKEIDV
SYVKIEEVIGAGEFGEVCRGRLKAPGKKESCVAIKTLKGGYTERQRREFLS
EASIMGQFEHPNIIRLEGVVTNSMPVMILTEFMENGALDSFLRLNDGQFTV
IQLVGMLRGIASGMRYLAEMSYVHRDLAARNILVNSNLVCKVSDFGLSRFL
EENSSDPTYTSSLGGKIPIRWTAPEAIAFRKFTSASDAWSYGIVMWEVMSF
GERPYWDMSNQDVINAIEQDYRLPPPPDCPTSLHQLMLDCWQKDRNARPRF
PQVVSALDKMIRNPASLKIVARENGGASHPLLDQRQPHYSAFGSVGEWLRA
IKMGRYEESFAAAGFGSFELVSQISAEDLLRIGVTLAGHQKKILASVQHMK
SQAKPGTPGGTGGPAPQY
SEQ ID NO: 2 is the amino acid sequence of human
serum albumin preproprotein (NP_000468.1).
Amino acid residues 25-609 encode the mature
peptide.
(SEQ ID NO: 2)
MKWVTFISLLFLFSSAYSRGVFRRDAHKSEVAHRFKDLGEENFKALVLIAF
AQYLQQCPFEDHVKLVNEVTEFAKTCVADESAENCDKSLHTLFGDKLCTVA
TLRETYGEMADCCAKQEPERNECFLQHKDDNPNLPRLVRPEVDVMCTAFHD
NEETFLKKYLYEIARRHPYFYAPELLFFAKRYKAAFTECCQAADKAACLLP
KLDELRDEGKASSAKQRLKCASLQKFGERAFKAWAVARLSQRFPKAEFAEV
SKLVTDLTKVHTECCHGDLLECADDRADLAKYICENQDSISSKLKECCEKP
LLEKSHCIAEVENDEMPADLPSLAADFVESKDVCKNYAEAKDVFLGMFLYE
YARRHPDYSVVLLLRLAKTYETTLEKCCAAADPHECYAKVFDEFKPLVEEP
QNLIKQNCELFEQLGEYKFQNALLVRYTKKVPQVSTPTLVEVSRNLGKVGS
KCCKHPEAKRMPCAEDYLSVVLNQLCVLHEKTPVSDRVTKCCTESLVNRRP
CFSALEVDETYVPKEFNAETFTFHADICTLSEKERQIKKQTALVELVKHKP
KATKEQLKAVMDDFAAFVEKCCKADDKETCFAEEGKKLVAASQAALGL

1-30. (canceled)

31. A method for treating prostate cancer in a patient, comprising administering to the patient a polypeptide agent that inhibits EphB4 or Ephrin B2 mediated functions, wherein the polypeptide agent is a monomeric ligand binding portion of the EphB4 protein and comprises a modification that increases serum half-life, and wherein the prostate cancer is refractory to an anticancer therapy selected from the group consisting of: androgen depletion therapy, androgen receptor (AR) targeted therapy, hormone depletion therapy, immunotherapy treatment, treatment with a chemotherapeutic agent, treatment using depleting antibodies to specific tumor antigens, treatment using agonistic, antagonistic, or blocking antibodies to co-stimulatory or co-inhibitory molecules (immune checkpoints), targeted treatment with an immunoconjugate, antibody-drug conjugate (ADC), or fusion molecule comprising a depleting antibody to specific tumor antigens tumor antigen and a cytotoxic agent, targeted treatment with a small molecule kinase inhibitor, treatment using surgery, treatment using stem cell transplantation, and treatment using radiation.

32. The method according to claim 31, wherein the polypeptide agent comprises a sequence selected from the group consisting of amino acids 1-197, 16-197, 29-197, 1-312, 16-312, 29-312, 1-321, 16-321, 29-321, 1-326, 16-326, 29-326, 1-412, 16-412, 29-412, 1-427, 16-427, 29-427, 1-429, 16-429, 29-429, 1-526, 16-526, 29-526, 1-537, 16-537 and 29-537 of SEQ ID NO: 1 (“sEphB4 polypeptide”) associated covalently or non-covalently with an albumin selected from the group consisting of a human serum albumin (HSA) (“sEphB4-HSA”) and bovine serum albumin (BSA) (“sEphB4-BSA”).

33. The method according to claim 32, wherein the sEphB4-HSA comprises residues 16-326 of SEQ ID NO: 1 directly fused to residues 25-609 of SEQ ID NO: 2.

34. The method according to claim 32, wherein the sEphB4-HSA comprises residues 16-537 of SEQ ID NO: 1 directly fused to residues 25-609 of SEQ ID NO: 2.

35. The method according to claim 31, wherein the cancer is refractory to treatment using androgen receptor (AR) targeted therapy.

36. The method according to claim 31, wherein the cancer is refractory to treatment using radiation.

37. The method according to claim 31, wherein the cancer is refractory to treatment using sipuleucel-T.

38. The method according to claim 31, wherein the cancer is refractory to treatment using radium-223.

39. The method according to claim 31, wherein the treatment further comprises co-administration of a second anti-cancer therapy, wherein the second anti-cancer therapy works in a synergistic manner with the polypeptide agent that inhibits EphB4 or Ephrin B2 mediated functions.

40. The method according to claim 34, wherein the anti-cancer therapy is selected from the group consisting of: androgen depletion therapy, AR targeted therapy, hormone depletion therapy, immunotherapy treatment, treatment with a chemotherapeutic agent, treatment using depleting antibodies to specific tumor antigens, treatment using agonistic, antagonistic, or blocking antibodies to co-stimulatory or co-inhibitory molecules (immune checkpoints), targeted treatment with an immunoconjugate, antibody-drug conjugate (ADC), or fusion molecule comprising a depleting antibody to specific tumor antigens tumor antigen and a cytotoxic agent, targeted treatment with a small molecule kinase inhibitor, treatment using surgery, treatment using stem cell transplantation, and treatment using radiation.

41. A method for treating prostate cancer in a patient, comprising: 1) determining whether one or more cancer cells from a patient expresses or overexpresses EphB4; and 2) if one or more cells expresses or overexpresses EphB4, administering an effective amount of an isolated polypeptide agent that inhibits EphB4 or Ephrin B2 mediated functions, wherein the polypeptide agent is a monomeric ligand binding portion of the EphB4 protein and comprises a modification that increases serum half-life, and wherein the cancer is refractory to an anticancer therapy selected from the group consisting of: androgen depletion therapy, androgen receptor (AR) targeted therapy, hormone depletion therapy, immunotherapy treatment, treatment with a chemotherapeutic agent, treatment using depleting antibodies to specific tumor antigens, treatment using agonistic, antagonistic, or blocking antibodies to co-stimulatory or co-inhibitory molecules (immune checkpoints), targeted treatment with an immunoconjugate, antibody-drug conjugate (ADC), or fusion molecule comprising a depleting antibody to specific tumor antigens tumor antigen and a cytotoxic agent, targeted treatment with a small molecule kinase inhibitor, treatment using surgery, treatment using stem cell transplantation, and treatment using radiation.

42. The method according to claim 41, wherein the polypeptide agent comprises a sequence selected from the group consisting of amino acids 1-197, 16-197, 29-197, 1-312, 16-312, 29-312, 1-321, 16-321, 29-321, 1-326, 16-326, 29-326, 1-412, 16-412, 29-412, 1-427, 16-427, 29-427, 1-429, 16-429, 29-429, 1-526, 16-526, 29-526, 1-537, 16-537 and 29-537 of SEQ ID NO: 1 (“sEphB4 polypeptide”) associated covalently or non-covalently with an albumin selected from the group consisting of a human serum albumin (HSA) (“sEphB4-HSA”) and bovine serum albumin (BSA) (“sEphB4-BSA”).

43. The method according to claim 42, wherein the sEphB4-HSA comprises residues 16-326 of SEQ ID NO: 1 directly fused to residues 25-609 of SEQ ID NO: 2.

44. The method according to claim 42, wherein the sEphB4-HSA comprises residues 16-537 of SEQ ID NO: 1 directly fused to residues 25-609 of SEQ ID NO: 2.

45. The method according to claim 41, wherein the cancer is refractory to treatment using androgen receptor (AR) targeted therapy.

46. The method according to claim 41, wherein the cancer is refractory to treatment using radiation.

47. The method according to claim 41, wherein the cancer is refractory to treatment using sipuleucel-T.

48. The method according to claim 41, wherein the cancer is refractory to treatment using radium-223.

49. The method according to claim 41, wherein the treatment further comprises co-administration of a second anti-cancer therapy, wherein the second anti-cancer therapy works in a synergistic manner with the polypeptide agent that inhibits EphB4 or Ephrin B2 mediated functions.

50. The method according to claim 49, wherein the anti-cancer therapy is selected from the group consisting of: androgen depletion therapy, AR targeted therapy, hormone depletion therapy, immunotherapy treatment, treatment with a chemotherapeutic agent, treatment using depleting antibodies to specific tumor antigens, treatment using agonistic, antagonistic, or blocking antibodies to co-stimulatory or co-inhibitory molecules (immune checkpoints), targeted treatment with an immunoconjugate, antibody-drug conjugate (ADC), or fusion molecule comprising a depleting antibody to specific tumor antigens tumor antigen and a cytotoxic agent, targeted treatment with a small molecule kinase inhibitor, treatment using surgery, treatment using stem cell transplantation, and treatment using radiation.


SEQUENCE LISTING (2 sequences)

SEQ ID NO: 1 - protein, 987 aa, Homo sapiens
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 2 - protein, 609 aa, Homo sapiens [sequence omitted]